(19) United States
(12) Patent Application Publication
(10) Pub. No.: US 2005/0139220 A1
(43) Pub. Date: Jun. 30, 2005

(54) Method and apparatus for ventilation / oxygenation during guided insertion of an endotracheal tube

(75) Inventor: Kent L. Christopher, Denver, CO (US)

Correspondence Address: DORR CARSON SLOAN & BIRNEY, PC, 3010 EAST 6TH AVENUE, DENVER, CO, 80206, US

(73) Assignee: Evergreen Medical Incorporated

(21) Appl. No.: 11/067,860

(22) Filed: Feb. 28, 2005

Related U.S. Application Data
(63) Continuation-in-part of application No. 10/115,224, filed on Apr. 2, 2002, now Pat. No. 6,860,264.
Application No. 10/115,224 is a continuation-in-part of application No. 09/767,272, filed on Jan. 22, 2001, now Pat. No. 6,568,388.
Application No. 09/767,272 is a continuation-in-part of application No. 09/707,350, filed on Nov. 6, 2000, now Pat. No. 6,543,446.
Application No. 09/707,350 is a continuation-in-part of application No. 09/411,610, filed on Oct. 1, 1999, now Pat. No. 6,405,725.
Application No. 09/411,610 is a continuation-in-part of application No. 08/974,864, filed on Nov. 20, 1997, now Pat. No. 5,964,217.
Application No. 08/974,864 is a continuation of application No. 08/607,332, filed on Feb. 26, 1996, now Pat. No. 5,694,929.
Application No. 10/115,224 is a continuation-in-part of application No. 09/908,380, filed on Jul. 18, 2001, now Pat. No. 6,668,821.
Application No. 09/908,380 is a continuation-in-part of application No. 09/840,194, filed on Apr. 23, 2001, now Pat. No. 6,634,354.
(60) Provisional application No. 60/252,347, filed on Nov. 20, 2000.

(51) Int. Cl. A61M 16/00; A62B 9/06

(52) U.S. Cl. 128/207.14; 128/200.26; 128/207.15

(57) ABSTRACT

A method for endotracheal intubation allows resuscitation of the patient to continue during intubation. A curved guide having a mask and a ventilation port is inserted into the patient's mouth and upper airway. The patient is initially resuscitated by supplying a flow of air/oxygen through the mask and simultaneously applying cardiac chest compressions. An endotracheal tube is inserted over the distal end of a fiber optic probe. Resuscitation continues without interruption while the fiber optic probe and endotracheal tube are advanced along the guide into the patient's airway, thereby allowing the physician to carefully guide the fiber optic probe and endotracheal tube to a position past the larynx while resuscitation continues.

### RELATED APPLICATIONS

The present application is a continuation-in-part of the Applicant's co-pending U.S. patent application Ser. No. 10/115,224, filed on Apr. 2, 2002, which is a continuation-in-part of U.S. patent application Ser. No. 09/767,272, filed on Jan. 22, 2001, now U.S. Pat. No. 6,568,388, issued on May 27, 2003, which is a continuation-in-part of U.S. patent application Ser. No. 09/707,350, filed on Nov. 6, 2000, now U.S. Pat. No. 6,543,446, issued on Apr. 8, 2003, which is a continuation-in-part of U.S. patent application Ser. No. 09/411,610, filed on Oct. 1, 1999, now U.S. Pat. No. 6,405,725, issued on Jun. 18, 2002, which is a continuation-in-part of U.S. patent application Ser. No. 08/974,864, filed on Nov. 20, 1997, now U.S. Pat. No. 5,964,217, issued on Oct. 12, 1999, which is a continuation of U.S. patent application Ser. No. 08/607,332, filed on Feb. 26, 1996, now U.S. Pat. No. 5,694,929, issued on Dec. 9, 1997. U.S. patent application Ser. No. 10/115,224 (cited above) is also a continuation-in-part of U.S. patent application Ser. No. 09/908,380, filed on Jul. 18, 2001, now U.S. Pat. No. 6,668,821, issued on Dec. 30, 2003, which is a continuation-in-part of U.S. patent application Ser. No. 09/840,194, filed on Apr. 23, 2001, now U.S. Pat. No. 6,634,354, issued on Oct. 21, 2003, which is based in part on, and claims priority to U.S. Provisional Patent Application Ser: No. 60/252,347, filed on Nov. 20, 2000.

### BACKGROUND OF THE INVENTION

1. Field of the Invention.

The present invention relates generally to the field of respiratory devices and methods. More specifically, the present invention discloses a method and apparatus for guiding insertion of an endotracheal tube while the patient continues to receive cardiopulmonary resuscitation in the form of artificial ventilation and cardiac compressions.

2. Statement of the Problem.

In emergency situations involving cardiopulmonary patients or other patients with compromised or arrested breathing, an oral airway is first inserted into the patient's mouth. A face mask is then placed over the patient's mouth and nose. The face mask is connected to an inflatable bag to maintain at least minimal oxygen flow to the lungs in the short term. This particular process of artificial ventilation is sometimes referred to as “bagging” the patient. It is suitable for initially stabilizing the patient. In order to breathe more effectively for the patient during cardiopulmonary resuscitation, and to prevent aspiration of stomach contents, an endotracheal tube (or ET tube) is placed into the trachea. Longer-term care usually requires continued artificial ventilation and attaching the patient to a ventilator (e.g., by means of the endotracheal tube). The transition from face mask to breathing through the endotracheal tube can be dangerous if insertion of the endotracheal tube takes too long, because the mask and oral airway must be removed and the flow of air/oxygen is interrupted while the endotracheal tube is inserted through the patient's mouth.

The typical conventional approach to making this transition involves discontinuing resuscitation and completely removing the mask and oral airway to expose the mouth. The physician inserts a rigid laryngoscope blade into the patient's mouth and then attempts to insert the endotracheal tube through the patient's mouth and upper airway and into the trachea in the conventional manner. Cardiac chest compressions are also discontinued during endotracheal tube insertion. The rigid laryngoscope blade is inserted into the mouth and advanced through the upper airway with an appropriate amount of force to distort the naturally curved airway so that the glottis is in straight alignment for direct visualization by the operator. Cardiac chest compressions are interrupted during this time because energy transmission from the vigorous cardiac chest compressions can cause an uncontrolled bouncing movement of the head and neck. Any movement of the head and neck impairs controlled manipulation of the laryngoscope for visualization and tube placement. Uncontrolled movement of the laryngoscope blade during forceful manipulation of the upper airway tissues can result in severe or life-threatening injury. Endotracheal intubation with the rigid laryngoscope blade may require a significant amount of time, even if the patient is motionless. The procedure is more difficult if the patient is less than completely cooperative and relaxed, or if the patient's airway has suffered trauma, or the tongue has fallen back to close the airway. The patient may not be breathing during this time, or may not be breathing sufficiently to maintain adequate blood oxygen levels, particularly if cardiac arrest is present. If the transition process takes more than a few seconds, the physician must temporarily abandon the effort and return to resuscitation by reinserting the oral airway and replacing the face mask, and then resuming cardiac chest compressions. The transition process may have to be repeated several times before the endotracheal tube is successful installed. In addition, the speed with which the transition process must be completed increases the chances of a mistake being made or unnecessary injury to the patient during the intubation procedure. Irreversible damage to vital organs such as the brain and heart can occur after 30 seconds of interruption of artificial ventilation, and in an even shorter time in the absence of cardiac chest compressions.

Endotracheal tubes are also used in semi-emergency situations to ventilate patients with respiratory failure who may be conscious or semi-conscious. The conventional approach requires the patient to lie still while the physician inserts a rigid laryngoscope blade into the patient's mouth and trachea. Delivery of ventilation and/or oxygen is also interrupted during this period. The endotracheal tube is then inserted into place while the laryngoscope blade keeps the patient's airway open. Successful intubation depends on the patient being cooperative and completely relaxed, which unfortunately is often not the case. Even with a cooperative patient, intubation is very uncomfortable and can cause the patient to panic due to the difficulty in breathing during the procedure. This procedure can also result in a choking or gagging response that can cause the patient to regurgitate and aspirate contents from the stomach. One conventional response to these shortcomings has been to sedate the patient during intubation. Tranquilizers make the patient more cooperative and less likely to choke during intubation, but also tend to suppress the patient's breathing and blood pressure. These side effects may be unacceptable when dealing with a patient who already suffers from shallow or irregular breathing or depressed blood pressure. A need exists for improved devices and methods to guide insertion of an endotracheal tube and ensure that the patient's airway is open, and that also allows the patient to continue to receive air/oxygen and cardiac chest compressions during the insertion process.

3. Prior Art.

A wide variety of devices that combine face masks with tubes for ventilation (e.g., endotracheal tubes) have been used in the past, including the following:

| Inventor | Patent No. | Issue Date |
|---|---|---|
| Teves | 5,348,000 | Sep. 20, 1994 |
| Don Michael | 5,339,808 | Aug. 23, 1994 |
| Jeshuran | 5,197,463 | Mar. 20, 1993 |
| Northway-Meyer | 4,848,331 | Jul. 18, 1989 |
| Kondur | 4,580,556 | Apr. 8, 1986 |
| Donmichael | 4,497,318 | Feb. 5, 1985 |
| Dryden | 4,256,099 | Mar. 17, 1981 |
| Buttaravoli | 3,809,079 | May 7, 1974 |
| Michael et al. | 3,683,908 | Aug. 15, 1972 |

Teves discloses a system for dispensing oxygen or anesthesia via an interchangeable face mask and nasal catheter.

Don Michael discloses a endotracheal-esophageal intubation device that includes a face mask (see, FIG. 2 of the Don Michael patent).

Jeshuran shows an anesthesia mask that is initially placed over the patient's mouth and nose as shown in FIG. 7 of the Jeshuran patent. A fiber optic is inserted through an endotracheal tube, and then through an opening in a two-piece core as shown in FIG. 9 of the Jeshuran patent. The fiber optic is advanced into the trachea. The head is then unscrewed and the core segments are disassembled to allow the endotracheal tube to be inserted through the mask, as shown in FIG. 2 of the Jeshuran patent. The fiber optic serves as a guide for insertion of the endotracheal tube. The fiber optic is then withdrawn and the endotracheal tube cuff is inflated, as shown in FIG. 8 of the Jeshuran patent. However, Jeshuran does not show a curved guide to direct insertion of the fiber optic probe. The physician is faced with the problem of navigating the fiber optic probe past the patient's tongue and along the airway.

Northway-Meyer discloses a device for pulmonary ventilation concurrent with fiber optic examination of the respiratory tract and tracheal intubation. In particular, Northway-Meyer discloses a face mask with a plurality of ports for ventilation and intubation of the patient, and curved guide for advancing an endotracheal tube.

Kondur discloses another example of an adapter that allows insertion of an endotracheal tube through the face mask and nose of the patient. Here again, no curved guide is provided.

Donmichael discloses an esophageal obturator for blocking aspiration of stomach fluids while the face mask is being used for ventilating the lungs.

Dryden discloses a two-tube resuscitation system. One tube is used to supply air to the trachea, while the other tube is used for aspiration or administering medication.

Buttaravoli discloses a resuscitator having a face mask with a curved tube for supplying air to the patient's airway.

Michael et al. disclose an apparatus for sealing a patient's esophagus and providing artificial respiration. The apparatus includes a mouth shield and a curved main tube.

In addition, the prior art includes several references involving intubating pharyngeal airways that have a curved central tubular member, including the following:

| Inventor | Patent No. | Issue Date |
|---|---|---|
| Parker | 5,339,805 | Aug. 23, 1994 |
| Augustine | 5,203,320 | Apr. 20, 1993 |
| Berman | 4,069,820 | Jan. 24, 1978 |
| Berman | 4,068,658 | Jan. 17, 1978 |
| Berman | 4,067,331 | Jan. 10, 1978 |
| Berman | 4,054,135 | Oct. 18, 1977 |

Parker discloses a curved guide for intubation of a patient's trachea or suctioning of the hypopharynx or esophagus.

Augustine discloses a-tracheal intubation guide with a curved forward end.

The Berman patents show an intubating pharyngeal airway having a side access for passage of a tube. The side opening can be expanded or closed by means of either a hinge on the opposite side wall of the tube or by a cap

Finally, the prior art in the field of laryngeal masks includes the following:

| Inventor | Patent No. | Issue Date |
|---|---|---|
| Holever | 4,240,417 | Dec. 23, 1980 |
| Bodai | 4,351,328 | Sep. 28, 1982 |
| Grimes | 4,416,273 | Nov. 22, 1983 |
| Brain | 4,509,514 | Apr. 9, 1985 |
| Brain | 4,995,388 | Feb. 26, 1991 |
| Brain | 5,241,956 | Sep. 7, 1993 |
| Brain | 5,249,571 | Oct. 5, 1993 |
| Brain | 5,282,464 | Feb. 1, 1994 |
| Brain | 5,297,547 | Mar. 29, 1994 |
| Brain | 5,303,697 | Apr. 19, 1994 |
| Brain | 5,305,743 | Apr. 26, 1994 |
| Brain | 5,391,248 | Feb. 21, 1995 |
| Brain | 5,355,879 | Oct. 18, 1994 |
| Brain | 5,584,290 | Dec. 17, 1996 |
| Brain | 5,632,271 | May 27, 1997 |
| Owens et al. | 5,642,726 | Jul. 1, 1997 |
| Brain | 5,682,880 | Nov. 4, 1977 |
| Brain | 5,711,293 | Jan. 27, 1998 |
| Pagan | 5,771,889 | Jun. 30, 1998 |
| Neame et al. | 5,871,012 | Feb. 16, 1999 |
| Brain | 5,878,745 | Mar. 9, 1999 |
| Neame et al. | 5,881,726 | Mar. 16, 1999 |
| Burden | 5,890,488 | Apr. 6, 1999 |
| Brain | 5,896,858 | Apr. 27, 1999 |
| Cook | 5,937,860 | Aug. 17, 1999 |
| Neame et al. | 5,979,445 | Nov. 9, 1999 |
| Pagan | 5,983,897 | Nov. 16, 1999 |
| Pagan | 6,003,514 | Dec. 21, 1999 |
| Pagan | 6,012,452 | Jan. 11, 2000 |
| Greenfield | 6,050,264 | Apr. 18, 2000 |
| Brain | 6,055,984 | May 2, 2000 |
| Brain | 6,079,409 | Jun. 27, 2000 |
| Pagan | 6,116,243 | Sep. 12, 2000 |

Holever discloses an adaptor to connect a ventilator to an endotracheal tube, while also permitting insertion of a suction tube.

Bodai discloses a system for simultaneous ventilation and endotracheal suctioning of a patient.

Grimes discloses a connector valve assembly for endotracheal tubes.

The Brain '514 patent discloses a laryngeal mask with a generally elliptical shape and a guide tube.

Brain '388 patent discloses a laryngeal mask with a soft flexible collar surrounding the lumen of the mask, and also having a drainage tube.

The Brain '956 patent discloses a laryngeal mask airway with concentric drainage for esophageal discharge.

The Brain '571 patent discloses a laryngeal clamp airway.

The Brain '464 patent discloses a combined laryngeal mask and reflectance oximeter.

The Brain '547 patent discloses a laryngeal mask with an inflatable cuff and a V-shaped posterior side.

The Brain '697 patent discloses a laryngeal mask with a rigid handle at the proximal end of the guide tube.

The Brain '743 and '248 patents disclose a molding process for producing laryngeal masks.

The Brain '879 patent discloses a laryngeal mask with inflatable ring and inflatable back cushion.

The Brain '290 patent discloses a laryngeal mask with electrodes.

The Brain '271 patent discloses a laryngeal mask with a gastric drainage feature.

The Brain '880 patent discloses a laryngeal mask with a removable stiffener that can be attached to the guide.

The Brain '293 patent discloses a forming tool for deflating a laryngeal mask, such as that shown in the Brain '547 patent, prior to insertion.

The Pagan '889 patent discloses a mask assembly having an inflatable ring and a diaphragm attached to a backing plate.

The '012 patent to Neame et al. discloses a laryngeal mask with an inflatable bag.

The Brain '745 patent discloses a gastro-laryngeal mask with an inflatable cuff and a back cushion to engage the back wall of the pharynx.

The '726 patent to Neame et al. discloses a laryngeal mask with a cuff formed by interlocking ribs.

Burden discloses a coupling device for placing a stethoscope and an endotracheal tube in gaseous communication.

The Brain '858 patent discloses a laryngeal mask with a hinged bar to elevate the epiglottis.

Cook discloses a laryngeal mask with an inflatable toroidal peripheral portion having a recessed front notch.

The '445 patent to Neame et al. discloses a method for manufacture of a laryngeal mask in which the edges of the cuff are heat-sealed.

The Pagan '897 patent discloses a laryngeal mask with cuffs attached on both sides of a plate. The plate also forms a leading tip.

The Pagan '452 patent discloses a laryngeal mask with an air line extending to a foam cuff. The cuff can be compressed for insertion by applying suction to the air line.

Greenfield discloses a laryngeal mask requiring an obdurator inserted into the tube.

The Brain '984 patent discloses an endotracheal tube having tapered, closed nose with a triangular cross-section and lateral openings.

The Brain '409 patent discloses a laryngeal mask having a specific geometry for the guide tube and mask.

The Pagan '243 patent discloses a laryngeal mask with a plate separating two separate semi-annular cuffs bonded to opposite sides of the plate.

4. Solution to the Problem.

None of the prior art references discussed above teach or suggest a system that enables the patient to continue to be resuscitated with continued artificial ventilation and cardiac chest compressions while being intubated. In particular, the present system allows the endotracheal tube to be inserted and connected to a ventilator without interrupting the flow of air/oxygen to the patient's lungs and without interrupting cardiac chest compressions.

### SUMMARY OF THE INVENTION

This invention provides a method and apparatus for guiding insertion of an endotracheal tube into a patient's trachea during resuscitation. A guide having a mask and a ventilation port is inserted into the patient's mouth and hypopharynx. The patient is initially resuscitated by supplying a flow of air/oxygen through the ventilation port. A series of cardiac chest compressions are also applied to the patient. An endotracheal tube is inserted over the distal end of a fiber optic probe. Resuscitation continues without interruption of cardiac chest compressions or ventilation while the fiber optic probe and endotracheal tube are advanced along the guide into the patient's airway. The direction of the distal tip of the fiber optic probe can be controlled by the physician and allows the physician to carefully guide the fiber optic probe and endotracheal tube to a position past the larynx while resuscitation continues. The fiber optic probe is then removed from within the endotracheal tube while leaving the endotracheal tube in place within the trachea. The cuff on the endotracheal tube is inflated and a ventilator is connected to the proximal end of the endotracheal tube to ventilate the patient. Alternatively, the patient can be manually ventilated by connecting a resuscitation bag to the proximal end of the endotracheal tube.

A primary object of the present invention is to provide a method and apparatus for guiding insertion of an endotracheal tube that does not require interruption of either cardiac chest compressions or artificial ventilation in the resuscitation process.

Another object of the present invention is to provide a method and apparatus for improving insertion of an endotracheal tube by helping to keep the patient's airway open, and also allowing the physician to guide the insertion process via the fiber optic probe.

Another object of the present invention is to provide a method and apparatus for instilling local anesthetic into the patient's airway and suctioning excess secretions prior to insertion of the endotracheal tube.

Another object of the present invention is to provide a method and apparatus for guiding insertion of an endotracheal tube that lessens the risk of injury, particularly during cardiac chest compressions, and reduces patient discomfort.

Yet another object of the present invention is to provide a device that enables the physician to instill anesthetic and/or suction secretions from the patient's mouth and airway as the device is inserted.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a front perspective view of the face mask assembly.

FIG. 2 is a rear perspective view of the mask assembly corresponding to FIG. 1.

FIG. 3 is a cross-sectional view of the mask assembly corresponding to FIG. 1.

FIG. 4 is a front view of the face mask port 23 showing the stretchable opening 24 closed.

FIG. 5 is a cross-sectional view of the mouth and airway of a patient after the mask 20 has been initially placed over the patient's mouth and nose with the curved guide 25 extending into the mouth, over the tongue 14, and into the hypopharynx 15 while cardiac chest compressions are administered.

FIG. 6 is a cross-sectional view of the mouth and airway of the patient corresponding to FIG. 5 after the fiber optic probe 30 and endotracheal tube 40 have been inserted through the face mask port 23 and advanced along the curved guide 25 to a position below the larynx 18.

FIG. 7 is a front view of the mask port 23 corresponding to FIG. 6 showing the fiber optic probe 30 and endotracheal tube 40 in cross-section.

FIG. 8 is a cross-sectional view of the mouth and airway of the patient corresponding to FIG. 5 after the fiber optic probe 30 has been removed from within the endotracheal tube 40.

FIG. 9 is a cross-sectional view of the mouth and airway of the patient corresponding to FIG. 5 showing the face mask 20 being removed while the endotracheal tube 40 remains in place.

FIG. 10 is a cross-sectional view of the mouth and airway of the patient corresponding to FIG. 5 after the mask 20 has been removed, the endotracheal tube cuff 44 has been inflated, and a ventilator 50 has been connected to the endotracheal tube 40.

FIG. 11 is a cross-sectional view of the face mask 20 and guide 25 in an alternative embodiment in which the curved guide 25 is configured as a oral airway that engages the posterior surface of the mask 20 surrounding the face mask port 23.

FIG. 12 is a rear detail view of locking mechanism 21 used to engage the curved guide 25 to the posterior surface of the mask 20.

FIG. 13 is a front perspective view of an alternative embodiment of the face mask assembly.

FIG. 14 is a cross-sectional view of the mask assembly corresponding to FIG. 13.

FIG. 15 is a side elevational view corresponding to FIGS. 13 and 14 showing the mask assembly 20 placed over the patient's mouth and nose.

FIG. 16 is a front perspective view of a removable resuscitation attachment 70 that can be connected to the ventilation port 62 of the face mask assembly.

FIG. 17 is a side view of the resuscitation attachment 70 and flexible tubing 80.

FIG. 18 is a detail side view of an alternative embodiment of the resuscitation attachment 70 in which the location of the oxygen port 76 has been placed below the filter and one-way valve.

FIG. 19 is an exploded perspective view of the guide cap assembly.

FIG. 20 is a cross-sectional view of the guide cap assembly corresponding to FIG. 19.

FIG. 21 is a cross-sectional view of the mouth and airway of a patient after the mask 20 has been initially placed over the patient's mouth and nose, and the curved guide 25 is being advanced along the patient's airway while administering a local anesthetic from the syringe 55.

FIG. 22 is a perspective view of the stabilizer 120 that can attached to the fiber optic probe of an endoscope.

FIG. 23 is a perspective view of the endotracheal tube cap 125 that can be used in conjunction with a stabilizer 120.

FIG. 24 is a cross-sectional view of the mouth and airway of a patient after the face mask 20 has been initially placed over the patient's mouth and nose, and the stabilizer 120 and endotracheal tube cap 125 have been used to advance the endotracheal tube 40 to a position below the larynx 18.

FIG. 25 is a front perspective view of a guide 25 with a laryngeal mask 130 and a rotatable collar 60 for delivery of air/oxygen through the guide 25.

FIG. 26 is rear perspective view of the guide 25 and laryngeal mask 130 corresponding to FIG. 25.

FIG. 27 is a cross-sectional view of the guide 25 and laryngeal mask 130 corresponding to FIG. 25 with the laryngeal mask 130 inflated.

FIG. 28 is a detail cross-sectional view of the distal portion of the guide 25 and laryngeal mask 130.

FIG. 29 is a front perspective view of another embodiment of the guide 25 and laryngeal mask 130 in which the ventilation port 62 is fixed relative to the guide 25.

FIG. 30 is a rear perspective view of the guide 25 and laryngeal mask 130 corresponding to FIG. 29.

FIG. 31 is a front perspective view of another embodiment of the guide 25 and laryngeal mask 130 without a ventilation port.

FIG. 32 is a top perspective view of a patient's airway showing the inlet to the larynx, esophagus, and epiglottis.

FIG. 33 is a cross-sectional view of a patient's airway after the guide 25 and laryngeal mask 130 have been initially inserted.

FIG. 34 is a cross-sectional view of the guide 25 and laryngeal mask 130 and the patient's airway corresponding to FIG. 33 after the mask 130 has been inflated.

FIG. 35 is a cross-sectional view of the patient's airway, guide 25, and laryngeal mask 130 corresponding to FIGS. 33-34 showing a syringe 55 connected to the guide cap 91 to squirt anesthetic through the guide 25 and into the patient's airway to lessen discomfort.

FIG. 36 is a cross-sectional view of the guide 25, laryngeal mask 130, and the patient's airway corresponding to FIGS. 33-35 after an endotracheal tube 40 has been inserted.

FIG. 37 is a cross-sectional view of the guide 25, laryngeal mask 130, and the patient's airway corresponding to FIGS. 33-36 after the endoscope probe 30 has been withdrawn from within the endotracheal tube 40.

FIG. 38 is a cross-sectional view of the guide 25, laryngeal mask 130, and the patient's airway corresponding to FIGS. 33-37 after the mask 130 has been deflated and the guide 25 has been removed, leaving the endotracheal tube 40 in place in the patient's airway.

FIG. 39 is a cross-sectional view of the patient's airway corresponding to FIGS. 33-38 after the cuff 44 of the endotracheal tube 40 has been inflated and the patient has been connected to a ventilator 50.

FIG. 40 is a cross-sectional view of the patient's airway corresponding to FIG. 33-39 in an alternative methodology in which the guide 25 is withdrawn over the endoscope probe 30 while leaving the endotracheal tube 40 in place in the patient's airway.

### DETAILED DESCRIPTION OF THE INVENTION

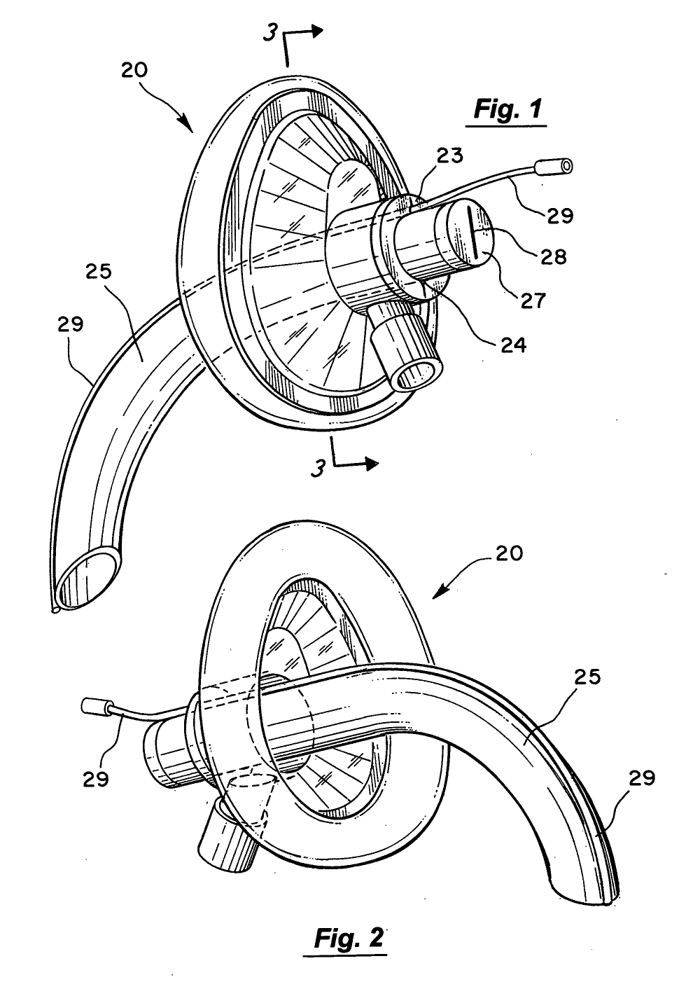

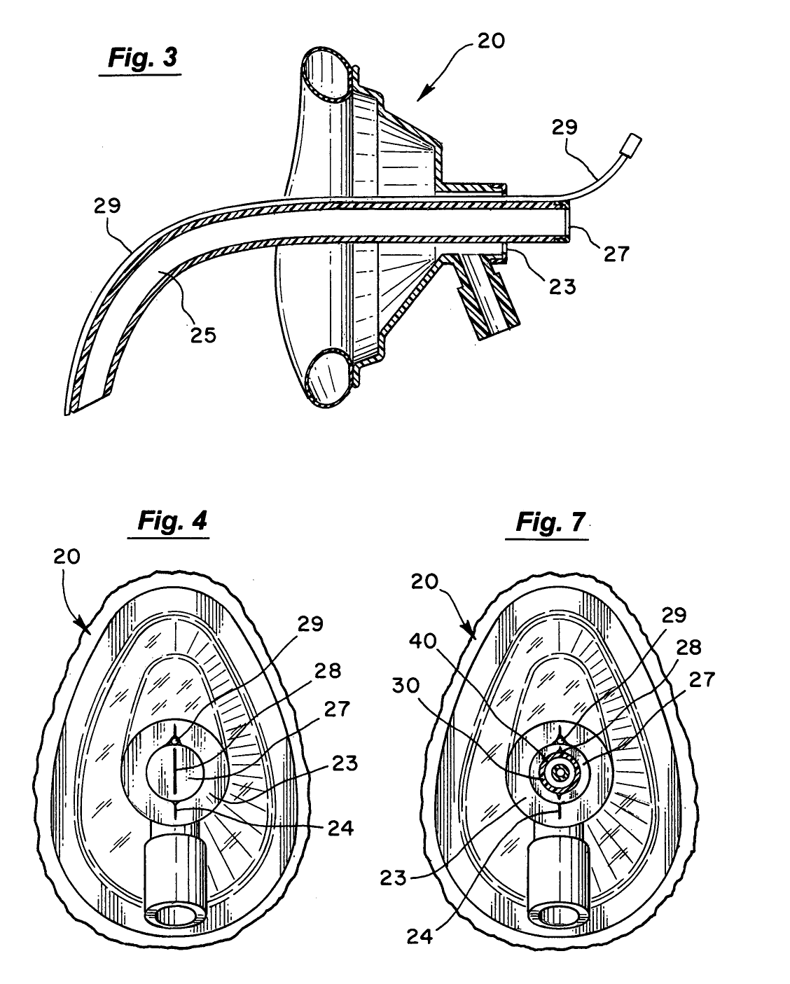

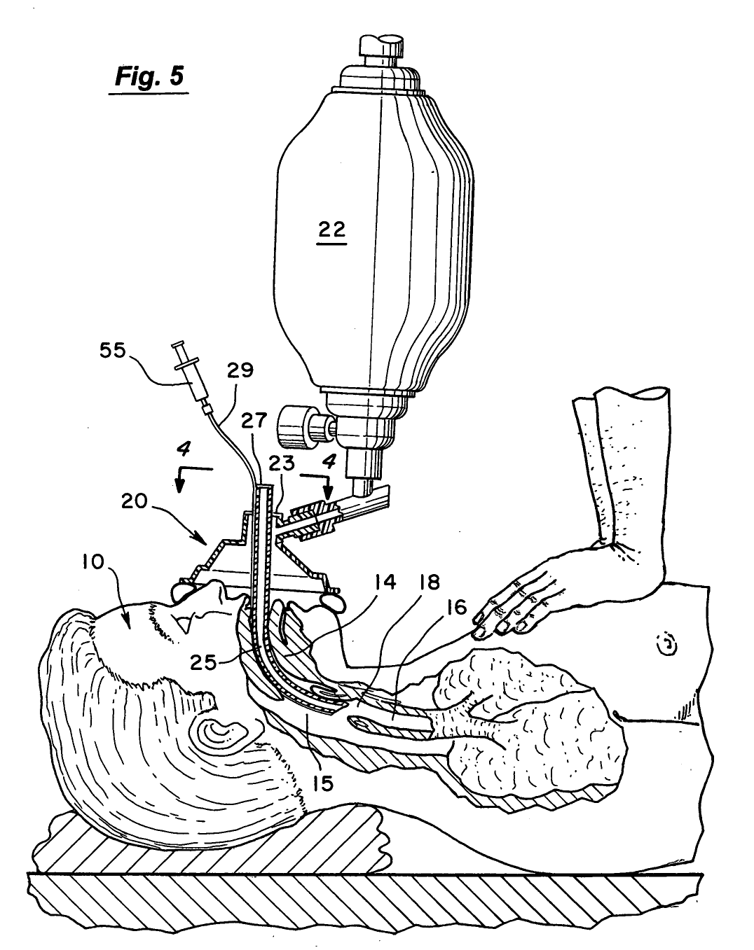

Face Mask Embodiment. FIGS. 1 through 24 show a first embodiment of the present invention using a face mask. Front and rear perspective views of this embodiment are illustrated in FIGS. 1 and 2. A corresponding cross-sectional view is shown in FIG. 3. The face mask 20 is adapted to fit over the patient's mouth and nose for resuscitation of the patient 10 as shown in FIG. 5. The mask 20 has a low profile and is made of an elastic material, such as rubber or flexible plastic, to allow the mask to conform to the contours of the patient's face and create a more air-tight seal around the mouth and nose.

The face mask 20 includes a resealable port 23. In the preferred embodiment, the face mask port 23 consists of a flexible, elastic membrane having a stretchable opening 24 with dimensions large enough to allow a curved guide 25 to pass through the face mask port 23. For example, this elastic membrane can be made of rubber with slot or hole forming an opening 24, as shown in FIG. 4.

As depicted in FIG. 5, the curved guide 25 can be readily inserted through the face mask port 23 while maintaining a substantially air-tight seal around the guide 25 to prevent gas from escaping from within the face mask 20. The guide 25 is generally tubular and includes a resealable port 27 at its proximal end. For example, the guide port 27 can be made of a flexible, elastic membrane having a stretchable slot or opening 28 with dimensions large enough to allow an endotracheal tube to pass through the guide port 27. The guide 25 extends posteriorly through the face mask 20 and has a curved distal portion that is inserted into the patient's mouth and hypopharynx 15 as the face mask 20 is placed over the patient's mouth. The distal portion of the curved guide 25 is generally J-shaped to follow the profile of a typical patient's airway through the mouth, over the tongue 14, and into the hypopharynx 15 just above the opening to the trachea 16. The guide 25 is shaped to be easily inserted along the normal anatomic curvature without forceful distortion of the upper airway, and to prevent the patient's tongue 14 and collapsible pharynx from obstructing access to the trachea 16, while also defining a channel for later insertion of an endotracheal tube. The guide 25 is typically made of plastic with sufficient strength and rigidity to keep the patient's teeth apart and prevent the patient from biting down on the endotracheal tube. The face mask port 23 allows the guide 25 to slide relative to the face mask 20, and also allows a limited range of rotation of the guide 25. This flexibility allows the guide 25 to accommodate a wide range of patient sizes and conditions.

In the preferred embodiment, the guide 25 is equipped with small tube 29 bonded to the exterior of the guide 25 that extends along the length of the guide 25 to its distal end. This tube 29 can be used to suction secretions from the patient's mouth and airway as the guide 25 is advanced. Alternatively a syringe 55 containing a local anesthetic (e.g., lidocaine or xylocaine) can be connected to the proximal end of the tube 29 to squirt anesthetic as the guide 25 is insert through the patient's mouth and into the hypopharynx 15, as illustrated in FIG. 5. If squirted with sufficient force, the anesthetic can be carried as far as the larynx 18 to deaden any discomfort associated with insertion of the endotracheal tube 40. Alternatively, the physician can squirt anesthetic directly down the main passageway of the guide 25. The main passageway can also be used for suctioning secretions from the patient's mouth and airway.

The patient is initially resuscitated by supplying a flow of air/oxygen through the mask. For example, the flow of air can be supplied by a resuscitation bag 22 attached to the mask 20 that is manually squeezed periodically to simulate natural breathing. However, other conventional air/oxygen supplies for resuscitation could be substituted at the connector for the face mask 20. In the preferred embodiment, the flow of oxygen/air from the resuscitation bag 22 is directed around the exterior of the curved guide 25. This tends to inflate the patient's mouth and airway, which distends the collapsible tissues, and thereby makes visualization and insertion of the endotracheal tube 40 easier.

A series of cardiac chest compressions can be applied to the patient's chest, as shown in FIG. 5, throughout this process to stimulate blood circulation. This is similar to conventional cardiopulmonary resuscitation (CPR). Studies have shown that continuing cardiac chest compressions can be critical. Even a relatively brief interruption of cardiac chest compressions can increase the risk of a negative outcome or neurological damage to the patient. Therefore, it is important to be able to continue cardiac chest compressions, as well as artificial ventilation, during the intubation process. Unlike the metal laryngoscope blades commonly found in the prior art, the guide 25 in the present invention does not create a risk of injury to the patient's airway if cardiac chest compressions continue during intubation.

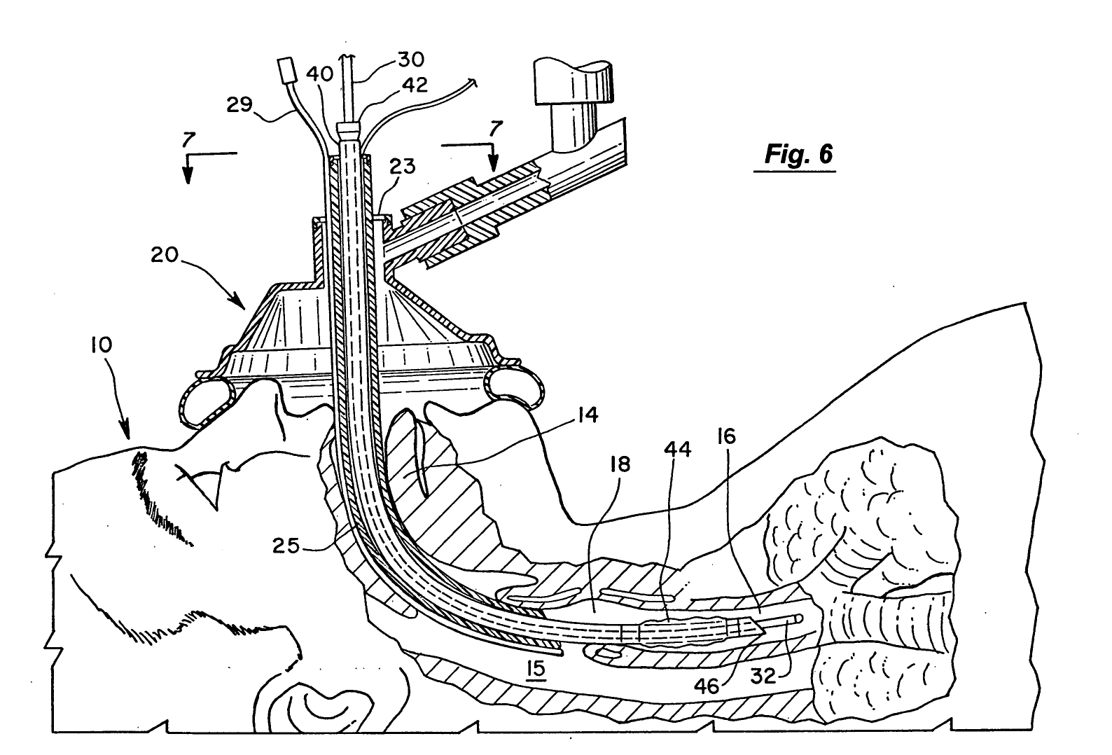

After the patient's condition has been stabilized to some degree during initial resuscitation, an endotracheal tube 40 is inserted over a fiber optic probe 30. The fiber optic probe 30 and endotracheal tube 40 are then inserted through the guide port 27 and along the guide 25 to a position within the trachea 16 past the larynx 18 while resuscitation continues, as illustrated in FIG. 6. The opening 28 in the flexible membrane stretches to allow the endotracheal tube 40 and fiber optic probe 30 to pass through the guide port 27, but maintains a sufficiently tight fit around the endotracheal tube 40 to prevent the escape of gas from within the mask 20, as shown in the front view of the face mask provided in FIG. 7.

The fiber optic probe 30 allows the physician to view within the patient's mouth and trachea 16 during insertion. Unlike the rigid laryngoscope blade, the fiber optic probe is flexible and can easily navigate curvatures. The physician can also remotely manipulate the direction of the probe tip 32 to control the direction of the fiber optic probe 30. The ability to easily steer the fiber optic probe, and the advanced optics and light source allow for adequate visualization, even during motion form cardiac chest compressions. The flexible tip of the fiber optic scope is designed to be atraumatic. These features minimize patient discomfort and risk of injury to the patient. The small size of the fiber optic probe 30 also allows the physician to thread the fiber optic probe 30 through relatively constricted areas within the airway, such, as the larynx 18. Most importantly, the fiber optic probe 30 and endotracheal tube 40 do not interfere with ongoing resuscitation of the patient and continued cardiac chest compressions.

The distal end 46 of the endotracheal tube 40 can beveled as illustrated most clearly in FIG. 6. Experience has shown that injury to the larynx 18 can be reduced by spinning the endotracheal tube 40 as it is advanced. The beveled end tends to keep the endotracheal tube 40 centered as it is passes through the vocal cords. Injury to the lining of the mouth and trachea can be reduced by using an endotracheal tube 40 made of a material having a low coefficient of friction, such as silicone. Bivona Medical Technologies of Gary, Indiana, markets a line of endotracheal tubes made of silicone with a helical reinforcing wire.

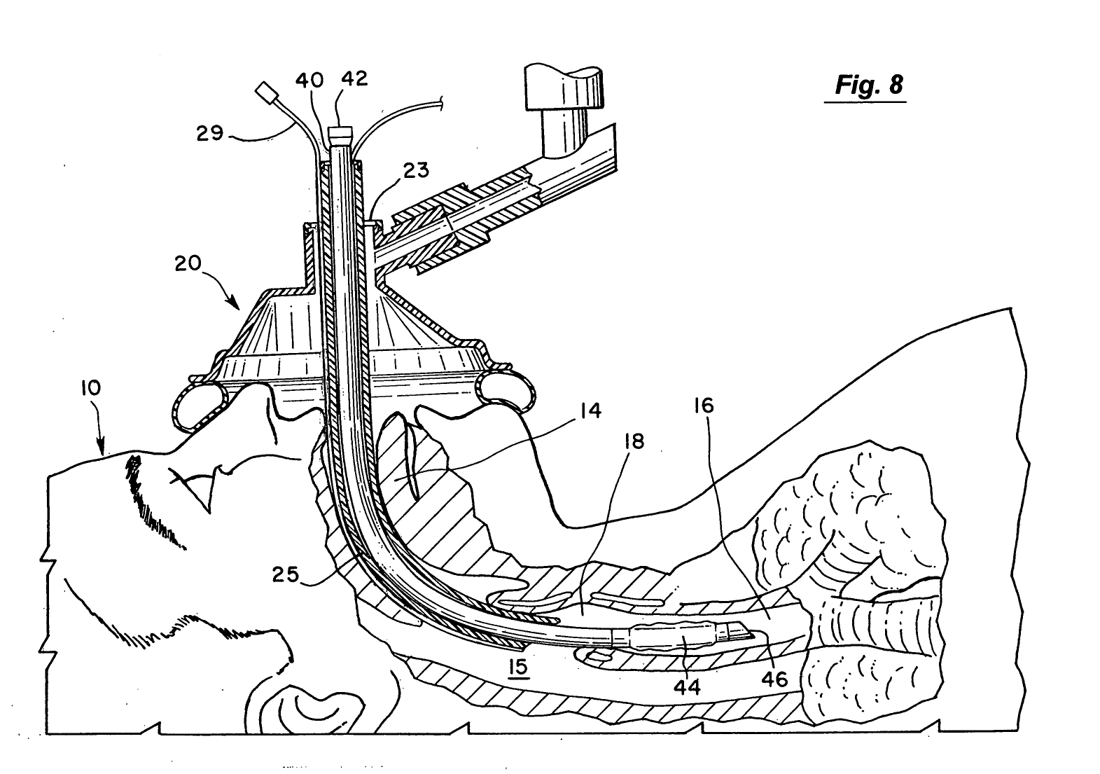

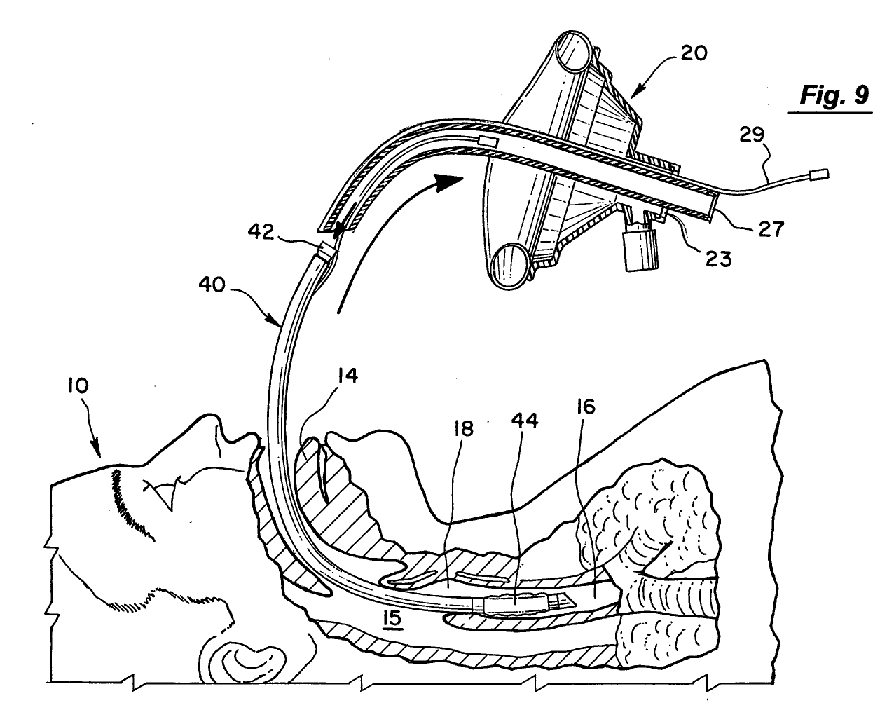

After the endotracheal tube 40 has been inserted, the fiber optic probe 30 is removed from within the endotracheal tube 40 through the proximal end of the endotracheal tube 40, as depicted in FIG. 8. The face mask 20 and guide 25 can then be removed while leaving the endotracheal tube 40 in place within the trachea 16, as shown in FIG. 9. The opening 28 in the flexible port 27 allows the face mask 20 and guide 25 to be withdrawn over the connector 42 at the proximal end of the endotracheal tube 40 with minimal effort and dislocation of the endotracheal tube 40. The position of the endotracheal tube 40 can be stabilized while the mask 20 is removed by manually gripping the proximal end of the endotracheal tube 40 and gradually urging it through the guide port 27 as the mask 20 is lifted from the patient's face. The physician can then reach under the face mask 20 to grip the endotracheal tube 40 after the mask 20 has been lifted sufficiently to allow access.

Alternatively, the face mask 20 can be removed while leaving the guide 25 in place to serve as an oral airway and to protect the endotracheal tube 40 from being bitten by the patient's teeth. After the face mask 20 has been removed, the endotracheal tube is taped to the patient's face, or held in place by some other suitable means for attachment.

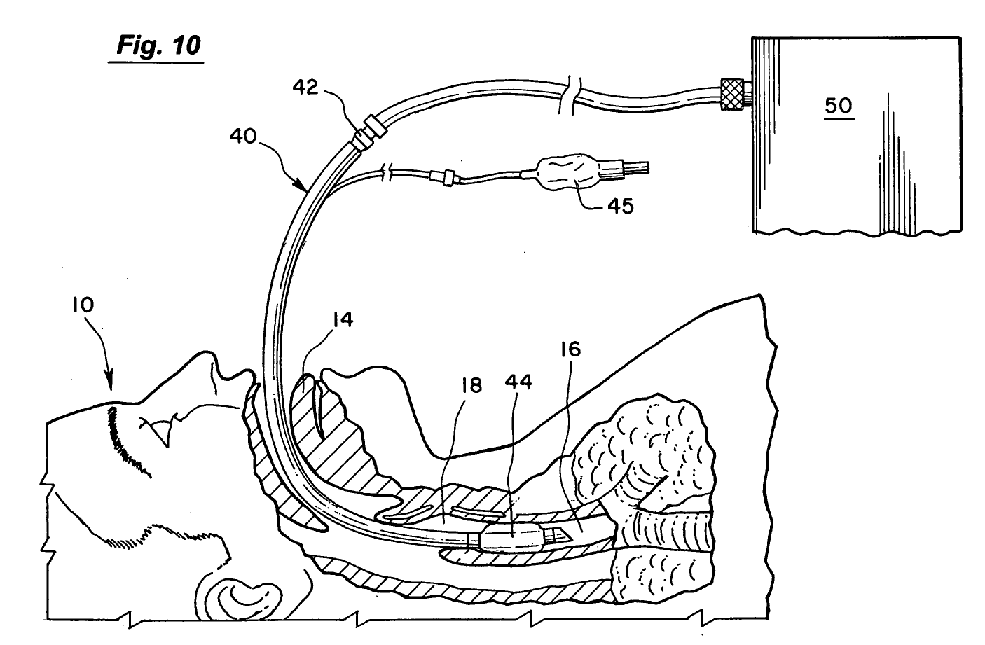

The cuff 44 at the distal end 46 of the endotracheal tube 40 is then inflated through the port valve 45 to block the trachea 16. An external ventilator 50 can be attached to the connector 42 at the proximal end of the endotracheal tube 40, as shown in FIG. 10. The patient can then be mechanically ventilated in the conventional manner via the endotracheal tube 40. Alternatively, the patient can be manually ventilated by attaching a resuscitation bag to the connector 42 at the proximal end of the endotracheal tube.

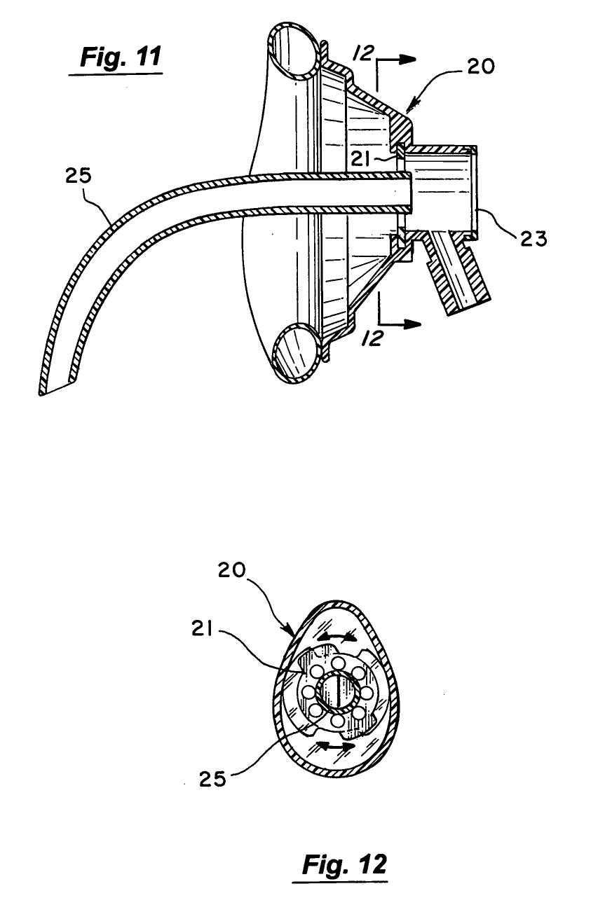

It should be understood that the guide 25 and mask 20 can have any number of possible embodiments. The embodiment shown in the FIGS. 1-9 uses a guide 25 that extends through an elastic port 23 in the face mask 20. This allows a limited range of motion between the guide 25 and mask 20 to make insertion of the guide easier, but requires two elastic ports 23 and 28. Alternatively, the guide 25 and mask 20 could be fabricated as two separate pieces that engage one another, as illustrated in FIG. 11. This eliminates the need for the guide port 27. In this embodiment, the guide 25 is separately inserted into the mouth, similar to a conventional oral airway. The mask 20 is then placed over the patient's mouth and nose so that the proximal end of the guide 25 engages a corresponding opening in the posterior face of the mask 20 to provide a relatively continuous passageway for insertion of the fiber optic probe 30 and endotracheal tube 40 through the face mask port 23 and along the guide 25. FIG. 12 provides a rear detail view of the locking mechanism 21 used to engage the guide 25 to the posterior face of the mask 20. The guide 25 can be readily disengaged by rotating it slightly relative to the face mask 20. After the endotracheal tube 40 has been inserted, the mask 20 is removed while leaving the guide 25 in place within the patient's mouth. The guide 25 remains around the endotracheal tube 40 and protects it from being bitten or crimped by the patient's teeth.

The guide 25 can consist of a J-shaped tubular member as shown in the drawings. The J-shaped tubular member is preferred as there are no edges to injure the airway. Alternatively, the distal portion of the guide 25 can have a U-shaped cross-section. The guide 25 can be molded from a suitable plastic material having a relatively low coefficient of friction to minimize irritation to the lining of mouth and trachea and to minimize resistance to insertion of the endotracheal tube 40 along the guide. Friction can be further reduced by applying a slippery coating to both the exterior and interior surfaces of the guide 25. A slippery coating can also be applied to the endotracheal tube to minimize friction between the endotracheal tube and the guide.

All of the components necessary to practice the present invention can be readily packaged as a kit for use in emergency rooms and intensive care units. The kit is sufficiently compact and inexpensive that it can be stocked on resuscitation carts widely used in hospitals, and carried in ambulances for use by emergency medical technicians in the field. The fiber optic probe can be operated using a battery-powered light source. The oxygen supply for the hospital or ambulance can be connected to the face mask 20 for resuscitation or to provide a flow of gas to the ventilator 50. The tube 29 extending along the guide 25 can also be connected to the suction system provided by the hospital or ambulance, if necessary.

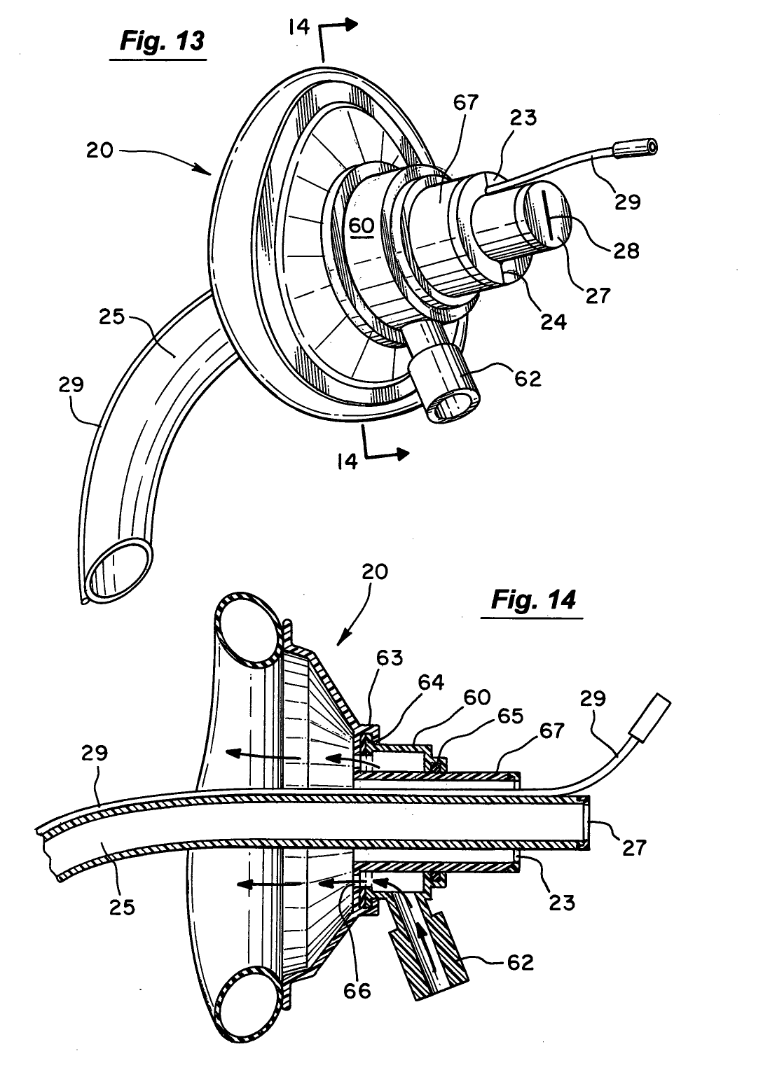

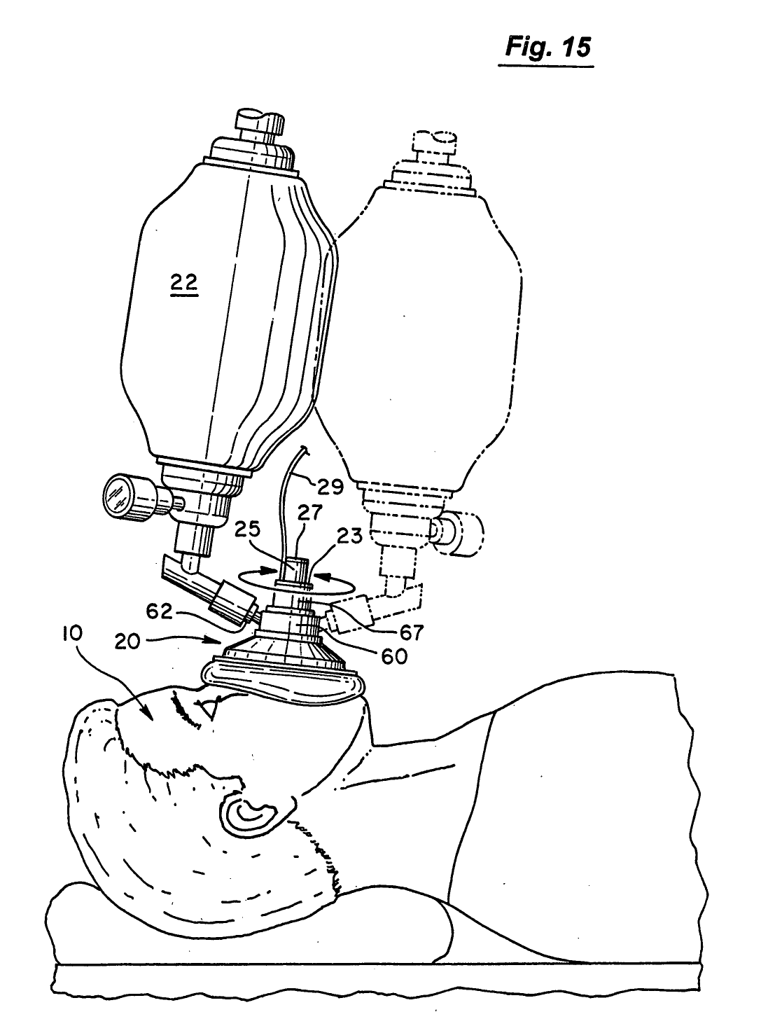

FIG. 13 is a front perspective view of an alternative embodiment of the face mask assembly with a rotating ventilation port. FIG. 14 shows a cross-sectional view of the mask assembly corresponding to FIG. 13. FIG. 15 is a side elevational view showing the mask assembly 20 placed over the patient's mouth and nose.

In contrast, the embodiment of the present invention illustrated in FIGS. 1-12 has a fixed ventilation port for connecting a resuscitation bag 22 or other source of air/oxygen to the face mask 20. This limitation may present a significant problem in emergency situations in which only limited access to the patient is available, or in which the patient cannot be readily moved. Similar problems can also occur in a hospital setting, due to the patient's position in bed, or surrounding medical equipment that can limit access to the patient from one side or the other.

Returning to FIGS. 13-15, the mask assembly includes a rotatable annular ventilation collar 60 with a ventilation port 62 that can be connected to a conventional respiration bag 22 or other air/oxygen source to ventilate the patient. The ventilation collar 60 allows the ventilation port 62 to be freely rotated to any desired orientation about the face mask port 23. Flexibility in the approach to the patient for artificial ventilation and intubation increases access of the individual performing simultaneous cardiac compressions.

Air from the resuscitation bag 22 flows through the ventilation port 62 and into the annular ventilation collar 60. It then flows through a plurality of small ventilation holes 66 in the mask 20 beneath the annular ventilation collar 60 into the patient's mouth and nose. The resuscitation bag 22 is typically used to initially resuscitate the patient, and to provide short-term ventilation until the endotracheal tube is in place and connected to a ventilator. After the patient has been intubated and connected to the ventilator, the resuscitation bag 22 can be removed. If needed, the resuscitation bag 22 can reconnected to the ventilation port 62 to supplement the flow provided by the ventilator.

In particular, the mask 20 includes a raised cylindrical flange 63 that engages a corresponding flange 64 extending around the base of the annular ventilation collar 60 to provide a rotatable, but generally air-tight seal between the mask 20 and the ventilation collar 60. A tubular member 67 extends upward from the surface of the mask 20 beneath the ventilation collar 60, and passes through the central opening in the annular ventilation collar 60. An O-ring 65 provides a rotatable, air-tight seal between the outer surface of the tubular member 67 and the ventilation collar 60, and also serves to retain the ventilation collar in place on the mask assembly 20.

A resealable face mask port 23 is provided at the upper opening of the tubular member 67, so that a curved guide 25 can be removably inserted through the face mask port 23 and into the patient's mouth and hypopharynx 15, as illustrated in FIG. 5. When the face mask port 23 is not in use (e.g., during initial resuscitation of a patient using the resuscitation bag 22), the face mask port 23 should remain sealed to prevent gas from escaping from the face mask 20. For example, the face mask port 23 can be a flexible membrane that has a stretchable opening to receive the guide 25. When the guide 25 is not inserted through the face mask port 23, the flexible membrane retracts to substantially seal the opening and prevent gas from escaping from the face mask port 23, as previously discussed. Alternatively, the face mask port 23 can be equipped with a removable cap to seal the port with it is not in use.

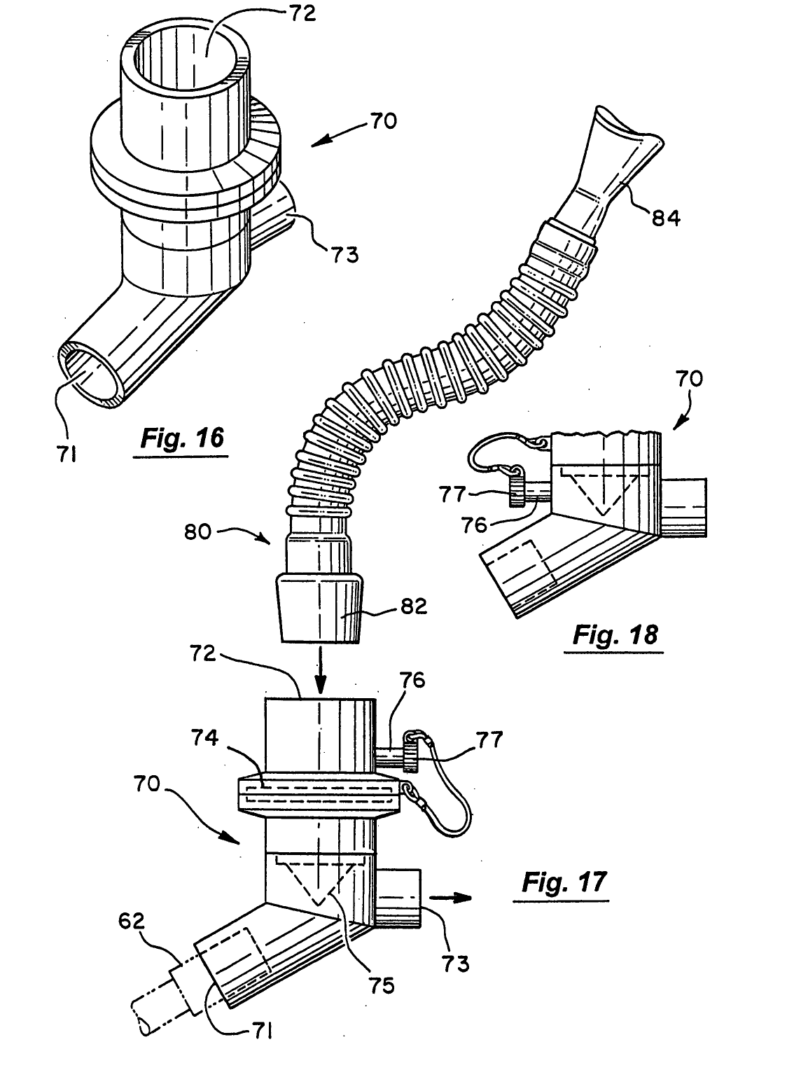

FIG. 16 is a perspective view of a removable resuscitation attachment 70 that can used in place of the resuscitation bag 22 for mouth-to-mask resuscitation by the rescue person. In a hospital setting, the first person responding to a patient in need of resuscitation typically activates an alarm to summon a resuscitation team, and then immediately begins mouth-to-mouth resuscitation of the patient until the resuscitation team arrives. To help minimize the risk of contamination, many hospitals equip each hospital bed with a face mask having a ventilation port for mouth-to-mask resuscitation. This type of face mask is also commonly provided for use by police and firemen with little medical training. When the resuscitation team arrives, this face mask is generally replaced with a system consisting of a second face mask, an oral airway, and a resuscitation bag. Since the patient usually requires intubation, this second face mask must be removed while an endotracheal tube is inserted into the patient's airway and the patient is connected to a ventilator. Each of these transitions entails an interruption in on-going cardiac chest compressions and artificial ventilation during resuscitation efforts, which can be detrimental to the patient. According to the American Heart Association, a period in excess of 30 seconds without breathing or circulation can cause irreversible brain and heart damage. Similarly, damage can occur if cardiac chest compressions are interrupted for much shorter periods.

In addition, the most common types of face masks used for initial resuscitation at the patient's bed do not include a guide or oral airway to keep the patient's airway open. As a result, initial efforts at manual resuscitation using the first face mask may be partially or completely ineffective, until the resuscitation team arrives and replaces the first face mask with a second face mask and a separate airway device used to keep the patient's airway open.

In contrast to the conventional approach practiced in many hospitals, as described above, the present invention allows the same face mask to be used throughout the entire process without interrupting resuscitation. In addition, the present invention includes a face mask with a curved guide that can be inserted into the patient's airway to maintain patency during the first effort to resuscitate the patient before the resuscitation team arrives.

Returning to FIG. 16, the resuscitation attachment 70 has an output port 71 that can be removably connected to the ventilation port 62 of the face mask 20. The healthcare provider administers mouth-to-mask resuscitation to the patient via the resuscitation attachment 70 and face mask 20.

The resuscitation attachment 70 includes an air filter 74 across the flow path between the input port 72 and output port 71, to help prevent the exchange of contaminants between the healthcare provider and patient. A one-way valve 75 (e.g., a duckbill valve) directs any backflow of air or contaminated fluids from the face mask 20 to the exhaust port 73, and thereby serves to further protect the healthcare provider from contaminants.

The healthcare provider can breathe directly into the input port 72 of the resuscitation attachment 70. Alternatively, a length of flexible tubing 80 can be connected to the resuscitation attachment 70 by means of a connector 82 that can be plugged into the input port 72 of the resuscitation attachment 70, as shown in FIG. 17. In the preferred embodiment, the flexible tubing 80 is approximately six inches in length and forms a helical coil for easier storage. The proximal end of the flexible tubing 80 has a mouthpiece 84 with an oval opening.

The resuscitation attachment 70 can also be equipped with an oxygen port 76, as shown in FIG. 17, that can be connected by tubing to a external oxygen source to supply supplemental oxygen to the patient through the flow path, in addition to the mouth-to-mask resuscitation provided by the healthcare provider. Each exhalation by the healthcare provider then carries oxygen-enriched air through the face mask 20 and into the patient's lungs. The oxygen port 76 can be closed with a removable cap 77 when the oxygen port 76 is not in use. The internal passageway within the flexible tubing 80 and resuscitation attachment 70 upstream from the one-way valve 75 serve as a reservoir for accumulation of oxygen between each exhalation by the healthcare provider.

FIG. 18 shows an alternative embodiment of the resuscitation attachment 70 with the oxygen port 76 placed below the one-way valve 75 and filter 74. In this embodiment, the internal passageway within the resuscitation attachment 70 downstream from the one-way valve 75 serves as a reservoir for accumulation of oxygen between each exhalation by the healthcare provider. The one-way valve 75 helps to prevent oxygen from escaping during the remainder of the resuscitation cycle. However, the exhalation port 73 prevents the build-up of excessive pressure that might be injurious to the patient's lungs.

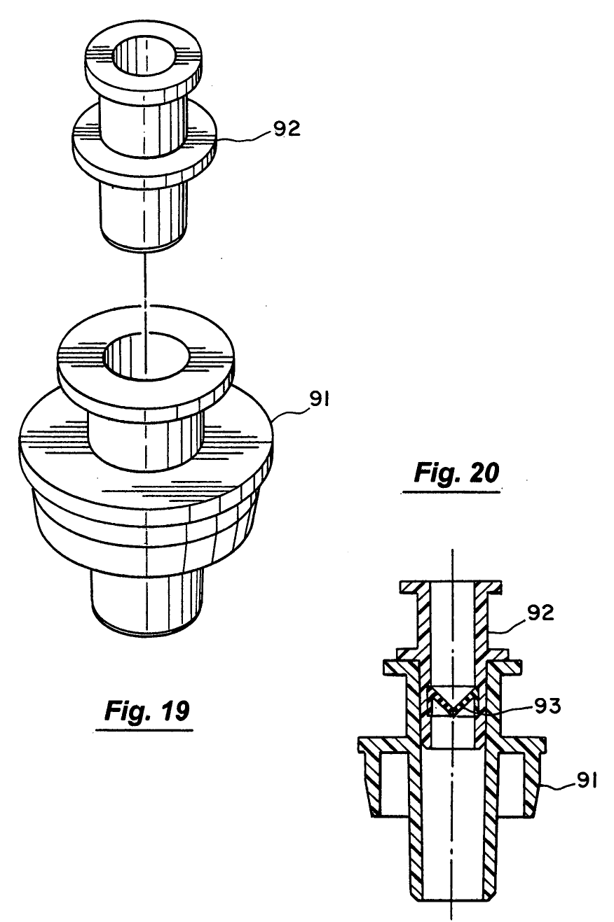

FIGS. 19-21 show a removable cap assembly that can be used to seal the proximal end of the tubular guide 25 in place of the guide port 27 shown for example in FIGS. 1, 4, and 7. As shown in the exploded perspective view of the cap assembly provided in FIG. 19, the guide cap 91 has an outside diameter dimensioned to seat into the proximal opening of the guide 25. A central passageway extends through the guide cap 91. As shown in the cross-sectional view provided in FIG. 20, a luer connector 92 with a one-way valve 93 (e.g., a duck-bill valve) is permanently attached to the guide cap 91 so that air or fluid can only flow down the passageway of the guide cap 91, but not up. Thus, the one-way valve 93 serves to prevent air/oxygen from escaping from within the face mask 20 during initial resuscitation.

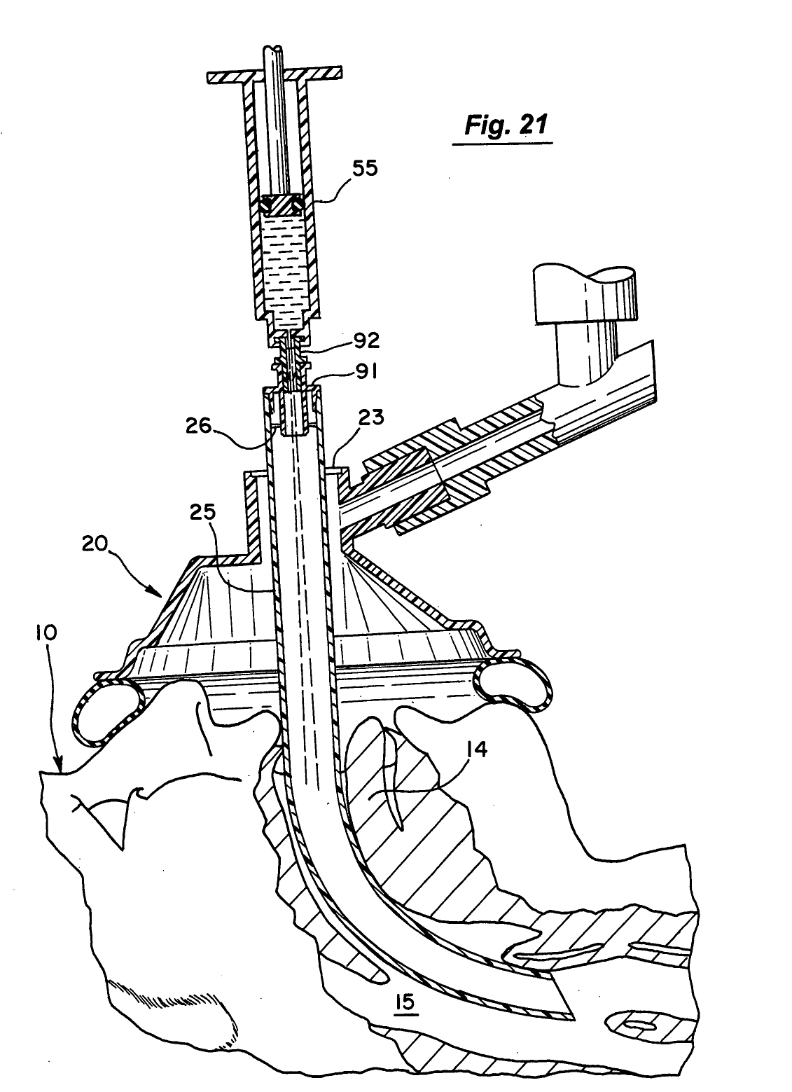

As illustrated in the cross-sectional view provided in FIG. 21, a syringe 55 containing anesthetic can be secured to the luer connector 92 on the guide cap 91. As the guide 25 is advanced into the patient's mouth and hypopharynx, the healthcare provider squirts anesthetic from the syringe 55, through the one-way valve 93 and guide 25 to lessen discomfort.

After the guide 25 has been advanced into position, the guide cap 91 is removed from the guide 25 to allow insertion of the endotracheal tube 40 through the guide 25, as previously discussed. An annular ring 26 within the proximal end of the guide 25 forms a loose seal around the endotracheal tube 40 to help prevent air/oxygen from escaping as the endotracheal tube 40 is being inserted.

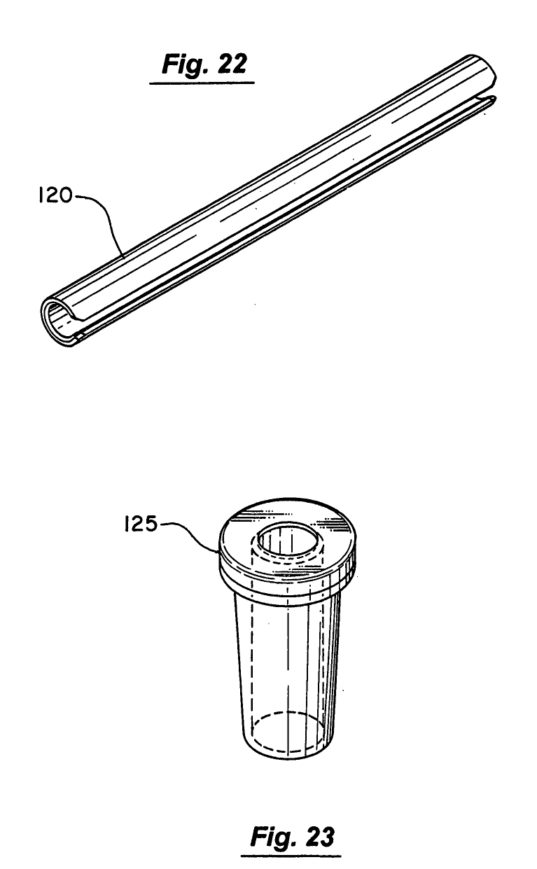

FIGS. 22-24 show another embodiment in which a stabilizer 120 is attached to the endoscope probe 30 and then used to advance the endotracheal tube 40 along the guide 25 and into the patient's trachea. In the preferred embodiment, the stabilizer 120 is a flexible plastic tube having a C-shaped cross-section, as shown in FIG. 22, that can be readily clipped over the fiber optic probe 30 at any desired location along its length.

The inside diameter of the stabilizer 120 should be selected to provide a snug, frictional fit against the exterior of the fiber optic probe 30 so that the stabilizer 120 will not readily slide after it has been attached to the fiber optic probe 30. The stabilizer 120 can also be readily removed from the endoscope probe 30 by the healthcare provider for cleaning or to adjust its location on the probe 30. The stabilizer 120 should have outside dimensions sufficiently large to push the endotracheal tube forward as the fiber optic probe 30 is advanced by the healthcare provider, and sufficiently small to fit through the face mask port.

The proximal end of the endotracheal tube 40 can be fitted with a removable cap 125 shown in FIG. 23. This cap 125 has outside dimensions selected so that it can be inserted snugly into the proximal opening of the endotracheal tube 40 and yet is sufficiently small to fit through the face mask port, if necessary.

A central passageway extends axially through the cap 125 to receive the fiber optic probe 30. The fiber optic probe 30 passes freely through the cap 125. However, the cap passageway has an inside diameter smaller than the stabilizer 120, so that the stabilizer 120 will abut and push against the proximal end of the endotracheal tube 40 as the fiber optic probe 30 is advanced by the healthcare provider.

In practice, this embodiment of the present invention typically uses the following sequence of steps. First, the face mask 20 is placed over the patient's mouth and the patient is initially resuscitated by a flow of air/oxygen delivered through the face mask ventilation port. With the guide cap 91 sealing the proximal end of the guide 25, the distal portion of the guide 25 is advanced by the healthcare provider into the patient's mouth and hypopharynx, as previously discussed. If necessary, a syringe 55 can be attached to the guide cap 91 to spray anesthetic down the guide 25 and into the patient's airway to less discomfort.

The stabilizer 120 is attached at a desired position on a fiber optic probe 30 of the endoscope. The fiber optic probe 30 is then inserted into the proximal end of the endotracheal tube 40 until the stabilizer 120 abuts the proximal end of the endotracheal tube 40. The location of the stabilizer 120 on the fiber optic probe 30 is normally selected so that the distal tip of the fiber optic probe 30 will extend slightly beyond the distal tip 46 of the endotracheal tube 40.

Optionally, a removable endotracheal tube cap 125 is attached to the proximal end of the endotracheal tube 40 prior to insertion of the fiber optic probe 30 so that the stabilizer 120 will push against this cap 125 as the healthcare provider advances the fiber optic probe 30. In this variation, the fiber optic probe 30 is inserted through both the endotracheal tube cap 125 and the endotracheal tube 40.

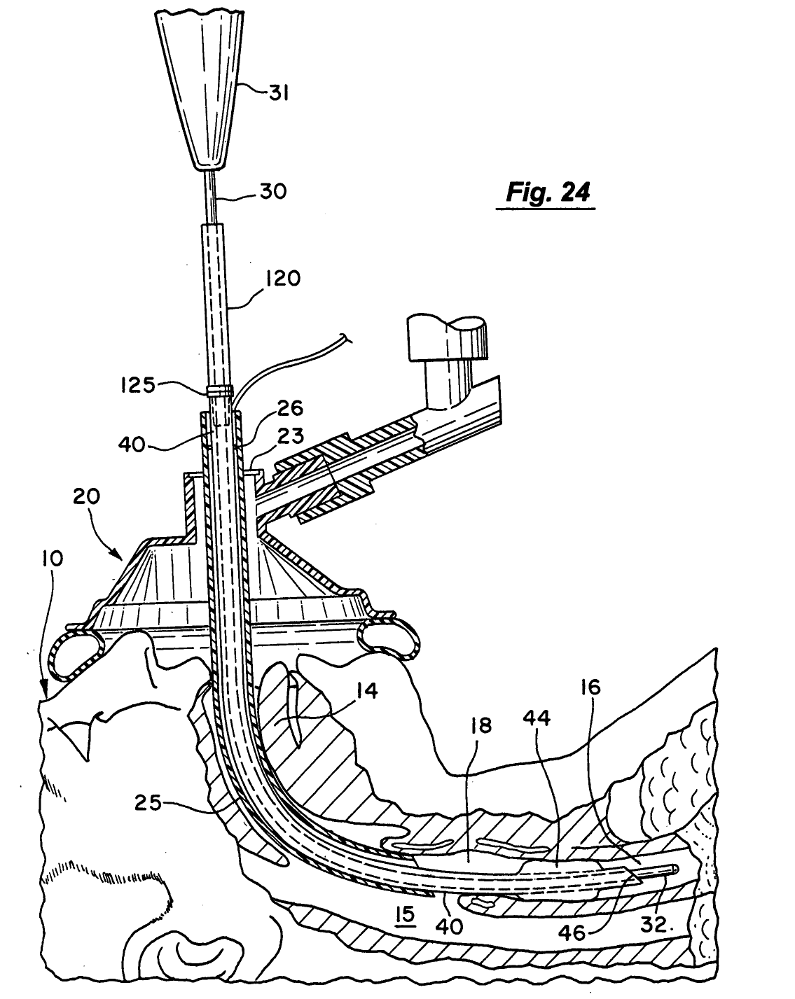

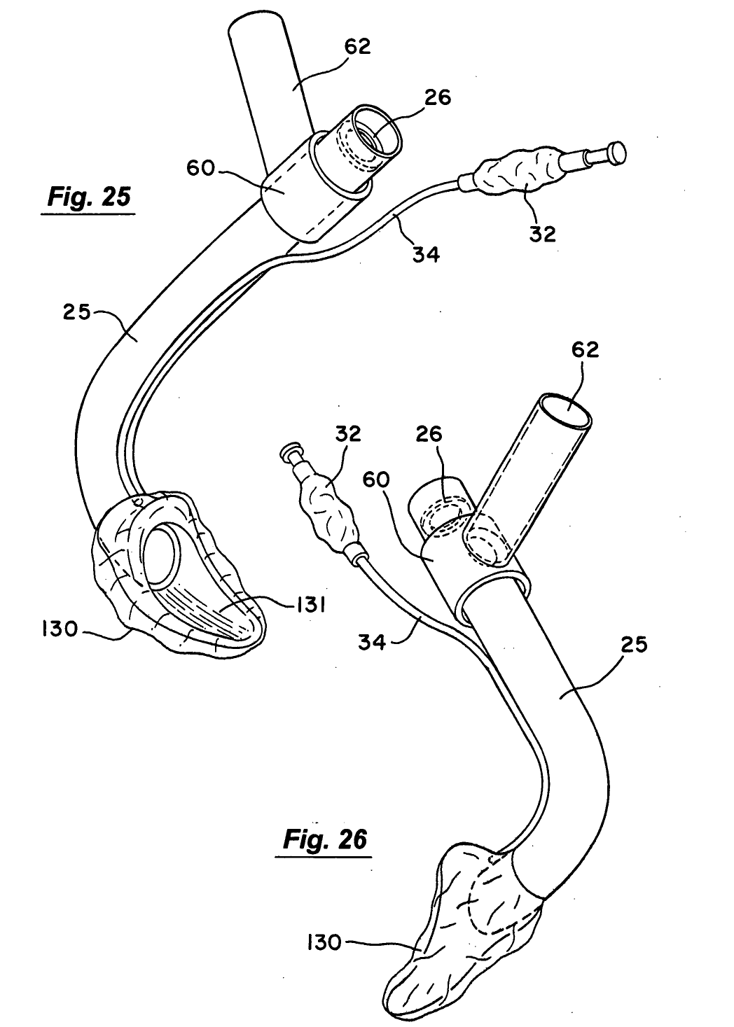

The guide cap 91 and syringe 55 are removed from the guide 25, and the assembly consisting of the endotracheal tube 40, fiber optic probe 30 and stabilizer 120 is inserted through the proximal end of the guide 25. The healthcare provider then pushes forward on the fiber optic probe 30 to advance the endotracheal tube 40 and the fiber optic probe 30 along the guide 25 and into the patient's trachea 16 as shown in FIG. 24. If the fiber optic probe 30 is part of a conventional endoscope, the healthcare provider can view through the endoscope probe 30 and manipulate the controls on the endoscope housing 31 to navigate the distal portion of the endotracheal tube 40 through the larynx and into the pharynx. Many conventional endoscopes include a suction channel extending the length of the fiber optic probe to its distal tip. This feature can be used to suction mucus or other secretions from the patient's airway as the endoscope/endotracheal tube assembly is inserted.

After the endotracheal tube 40 has been moved into position with its distal end in the trachea, the face mask 20 is removed over the proximal end of the endotracheal tube 40 while leaving the endotracheal tube 40 and fiber optic probe 30 in place. More specifically, the face mask 20 and guide 25 can either be removed together, or the face mask 20 can be remove first followed by the guide 25.

Before removing the face mask 20 and guide 25, the healthcare provider may wish to slide the stabilizer 120 a few centimeters toward the distal end of the fiber optic probe 30. This allows the endoscope to be pulled back relative to the endotracheal tube 40, so that the distal tip of the endoscope is located within the distal end of the endotracheal tube 40 and offers a view of both the endotracheal tube's distal tip and the patient's trachea. This enables the healthcare provider to monitor the position of the endotracheal tube 40 relative to the trachea as the face mask 20 and guide 25 are removed, as described above.

The fiber optic probe 30 is then withdrawn from within the endotracheal tube 40 and the endotracheal tube cap 125 is removed if one is present. Finally, the patient can be ventilated via a conventional ventilator connected to the endotracheal tube 40. Cardiac chest compressions can continue along with artificial ventilation through the entire intubation process.

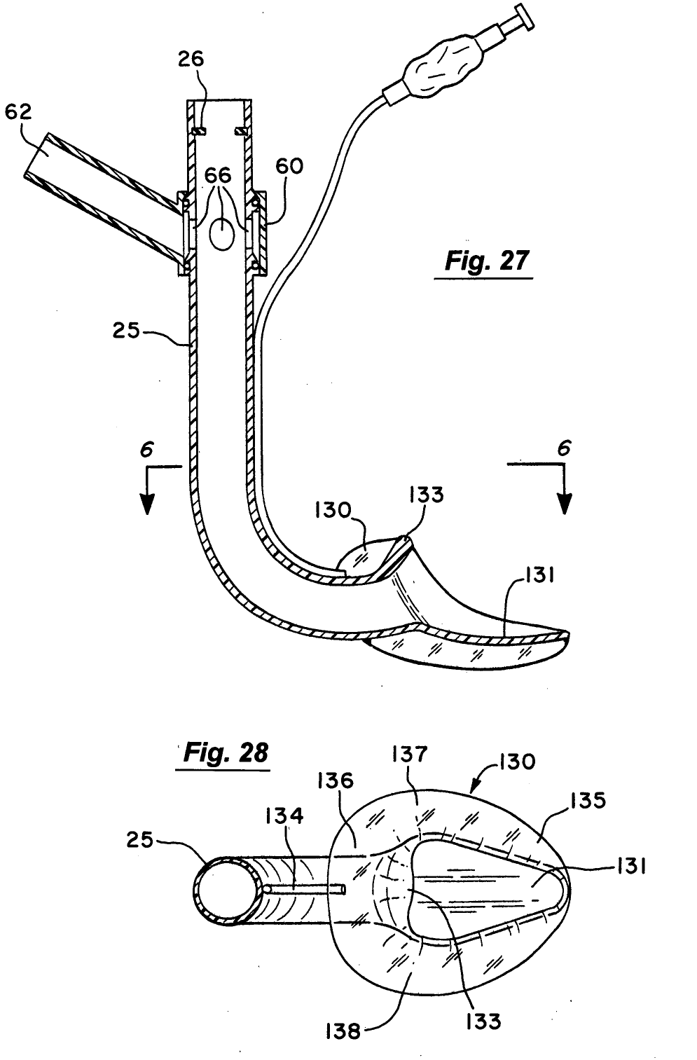

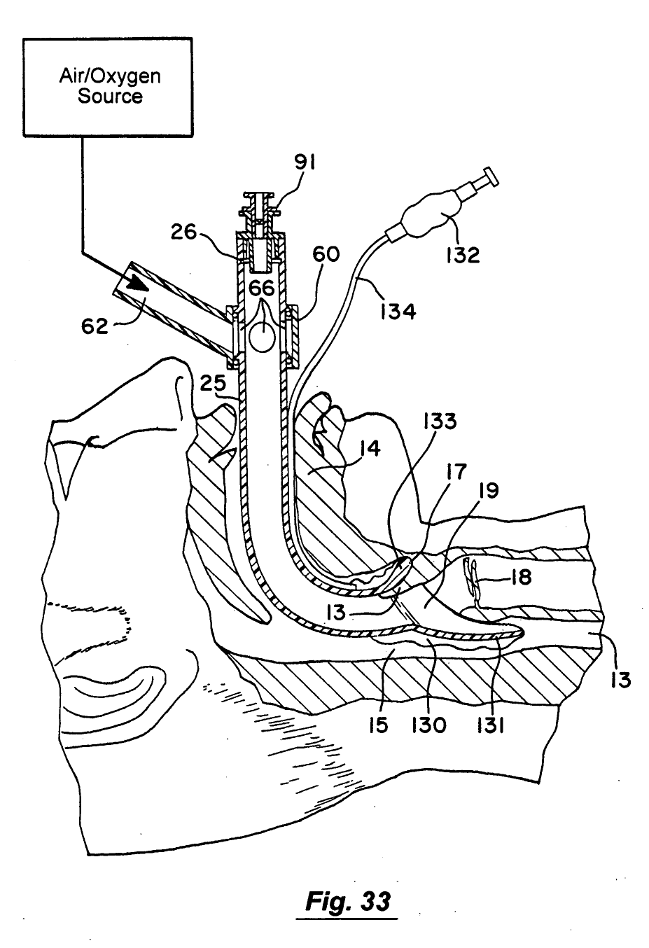

Pharyngeal Mask Embodiment. Turning to FIGS. 25 and 26, front and rear perspective views are provided of an alternative embodiment of the present invention using a laryngeal mask 130 in place of a face mask to ensure that the flow of air/oxygen delivered via the ventilation port 62 is delivered into the patient's lungs. This embodiment includes a tubular guide 25 with a laryngeal mask 130 surrounding its distal end. FIG. 27 is a corresponding cross-sectional view of the guide 25 with the laryngeal mask 130 inflated. FIG. 28 is a detail end view of the laryngeal mask 130 and the distal portion of the guide 25. The size and shape of the guide 25 are selected so that its distal portion can be readily inserted into the patient's mouth and upper airway with the laryngeal mask 130 substantially sealing the glottis 19, as shown in FIGS. 33-37. The proximal end of the guide 25 remains outside of the patient's mouth and therefore is accessible to the healthcare provider.

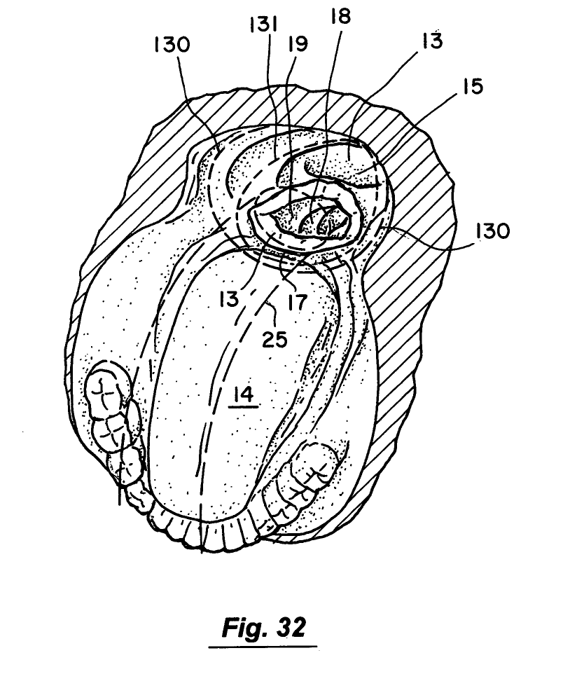

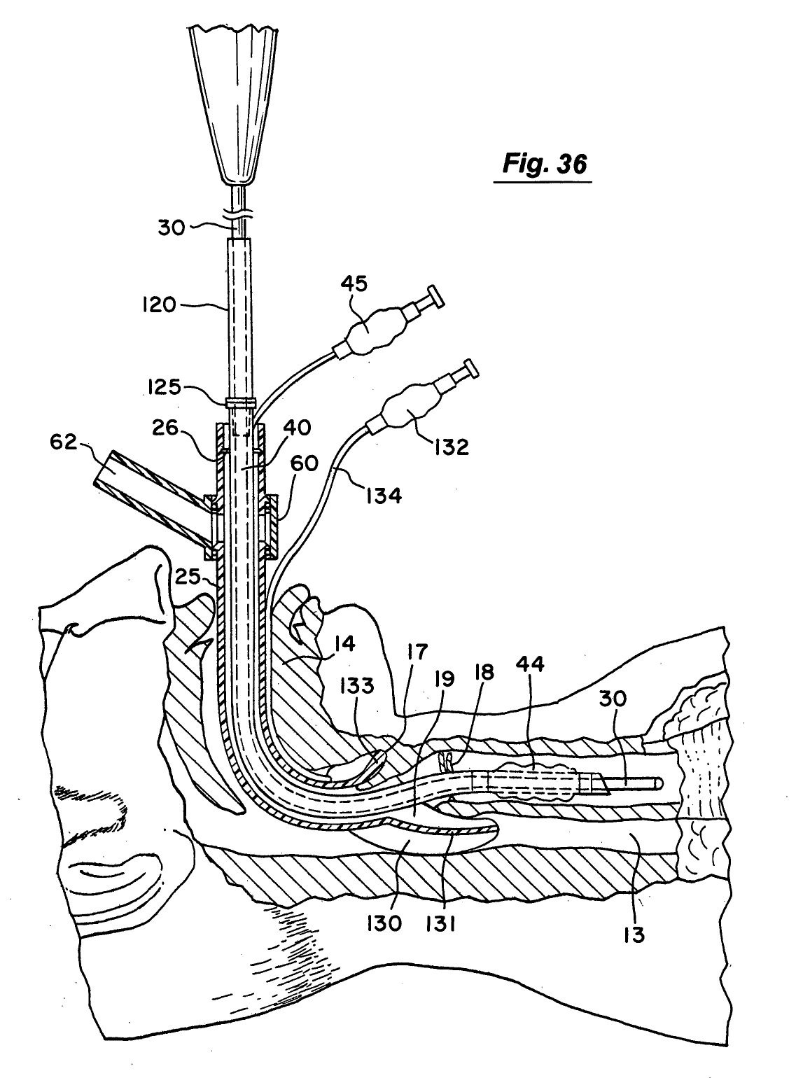

As before, the guide 25 is generally J-shaped to follow the profile of a typical patient's airway through the mouth, over the tongue 14, and into the laryngopharynx 15 just above the opening to the larynx 18 (see FIGS. 32 and 33). The guide 25 is shaped to prevent the patient's tongue 14 and collapsible pharynx from obstructing access to the trachea, while also defining a channel for later insertion of an endotracheal tube. The previously discussed safety and visualization benefits during concurrent cardiac chest compressions, as described above, apply to guide 25 of this invention. The guide 25 is typically made of plastic with sufficient strength and rigidity to keep the patient's teeth apart and prevent the patient from biting down on the endotracheal tube. This flexibility allows the guide 25 to accommodate a wide range of patient sizes and conditions. The inside diameter of the guide 25 should be sufficiently large to allow an endotracheal tube 40 to freely pass through the guide 25, as shown for example in FIG. 36, with extra room to allow air/oxygen to flow through the guide 25 around the endotracheal tube 40. Preferably, the distal opening of the guide 25 is beveled to substantially match the angle of the glottis 19 after insertion of the guide 25 into the patient's airway.

The laryngeal mask 130 consists a central support member 131 extending outward from the guide 25 to an inflatable member as illustrated in FIGS. 25-28. The laryngeal mask 130 is preferably made of a soft, flexible material (e.g., a polymer or rubber) to enable it to be advanced into position without injury to the patient and to create a substantially air-tight seal about the glottis 19. The degree of inflation of the laryngeal mask 130 can be adjusted through a small inflation tube 134 and air valve 132. Alternatively, the laryngeal mask 130 can be a cushion made of a soft, spongy material that is not inflatable. The laryngeal mask 130 and its support member 131 are shaped to meet several requirements. The lower portion 135 of the laryngeal mask 130 substantially blocks the esophagus to minimize the risk of regurgitation of stomach contents and the passage of air into the stomach. The upper portion 136 of the laryngeal mask 130 guides the distal end of the guide 25 into alignment with the glottis 19 as the guide is inserted along the patient's airway.

In the embodiment shown in the drawings, the laryngeal mask 130 is generally boot-shaped when inflated. The lower portion 135 of the laryngeal mask 130 forms the toe of the boot, which blocks the esophagus. The lower portion 135 of the laryngeal mask 130 also helps to align the distal opening of the guide 25 with the patient's glottis 19. After the mask 130 is inflated, the upper portion 136 of the mask 130 substantially fills the laryngopharynx 15 at the level of the glottis 19. The upper portion 136 of the laryngeal mask 130 surrounds the glottis 19 so that the distal opening of the guide 25 is sealed in fluid communication with the glottis 19. Thus, substantially all of the gas inhaled or exhaled by the patient passes through the guide 25. For example, the laryngeal mask 130 can be formed by injection blow molding, rotational molding, or dip molding.

In particular, the upper portion 136 of the mask 130 surrounding the distal opening of the guide 25 is canted at an angle to complement the natural angle of the glottis 19. The distal end of the guide 25 can also be beveled at this complementary angle. This enables the laryngeal mask 130 to directly engage the glottis 19 along the longitudinal axis of the patient's airway as the guide 25 is advanced. The shape of the upper portion 136 of the laryngeal mask 130 further helps to guide the distal opening of the guide 25 so that it is axially aligned with the glottis 19 and abuts the glottis 19 in an end-on relationship as the guide is inserted along the patient's airway. In contrast, conventional laryngeal masks typically approach the glottis 19 from a posterior or inferior position.

In the embodiment depicted in FIGS. 25-28, the proximal end of the guide 25 can be sealed by a removable guide cap 91 as shown in FIG. 19, 20, and 33-35. FIG. 33 is a cross-sectional view of a patient's airway after the guide 25 has been initially inserted. As shown in FIG. 33, the guide cap 91 has an outside diameter dimensioned to seat into the proximal opening of the guide 25 and thereby prevent the escape of gas through this opening. When inserted, the guide cap 91 abuts and seals against an annular seal ring 26 within the guide 25 as illustrated in FIG. 33. The guide cap 91 has a small passageway or port extending vertically through the guide cap 91. As shown in FIG. 20, a luer connector 92 with a one-way valve 93 (e.g., a duck-bill valve) is permanently attached to the guide cap 91 so that air or fluid can only flow down the passageway of the guide cap 91, but not up. Thus, the one-way valve 93 serves to prevent air/oxygen from escaping through the guide 25 during resuscitation.

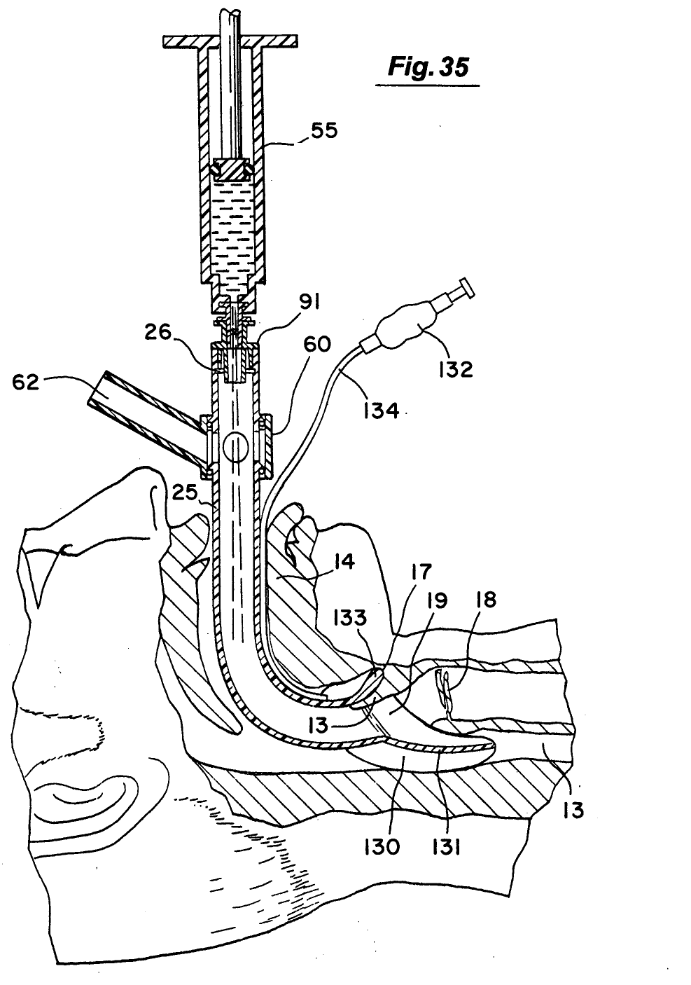

As illustrated in FIG. 35, a syringe 55 containing anesthetic can be secured to the luer connector 92 on the guide cap 91. As the guide 25 is advanced into the patient's mouth and hypopharynx, the healthcare provider squirts anesthetic from the syringe 55, through the one-way valve 93 and guide 25 to lessen discomfort. After the guide 25 has been advanced into position, the guide cap 91 is removed from the guide 25 to allow insertion of an endotracheal tube 40 and fiber optic probe 30 through the guide 25, as will be discussed below.

A flow of air/oxygen is delivery to the patient via the guide 25 through a ventilation port 62 extending at an angle from the side of the guide 25. A rotatable collar 60 allows the ventilation port 62 to be rotated about the central axis of the guide 25 to any desired orientation. Air/oxygen flows through the ventilation port 62 into the annular space between the collar 60 and the guide 25, and through a series of ventilation holes 66 into the interior of the guide 25, as shown in greater detail in FIG. 27. For example, the ventilation port 62 can be connected to a conventional ventilator or a resuscitation bag. Alternatively, a mouthpiece can be connected to the ventilation port 62 for initial patient resuscitation by a healthcare provider, as discussed above.

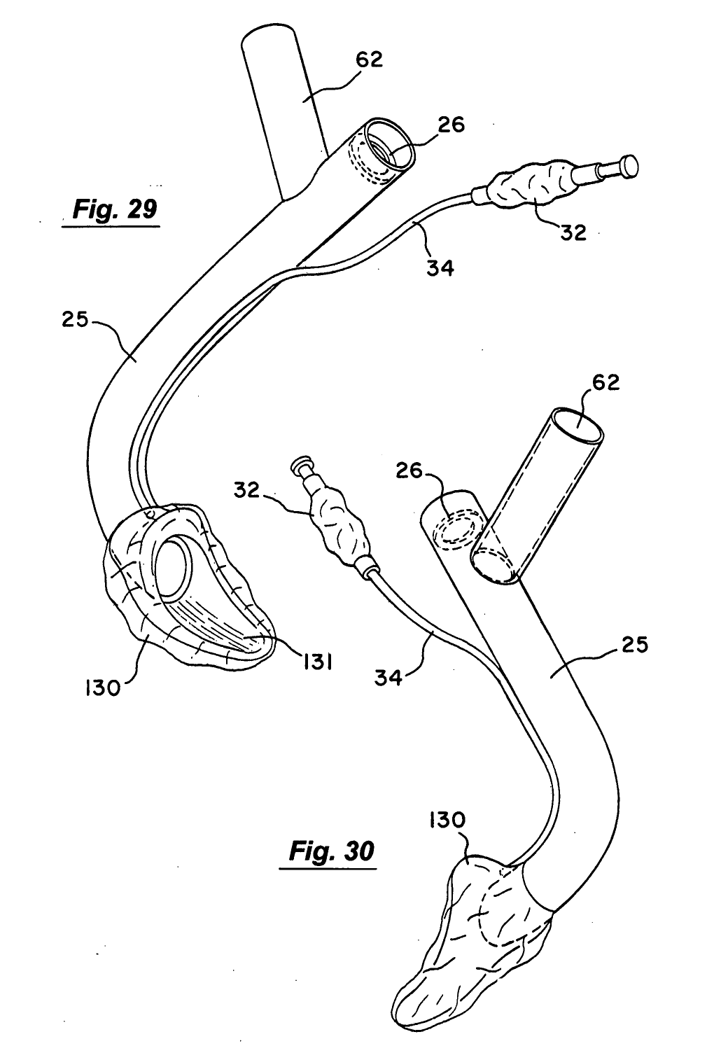

FIGS. 29 and 30 are front and rear perspective views of another embodiment of the present device in which air/oxygen is introduced directly into the guide 25 through a fixed ventilation port 62. This embodiment would be simpler and less expensive to build.

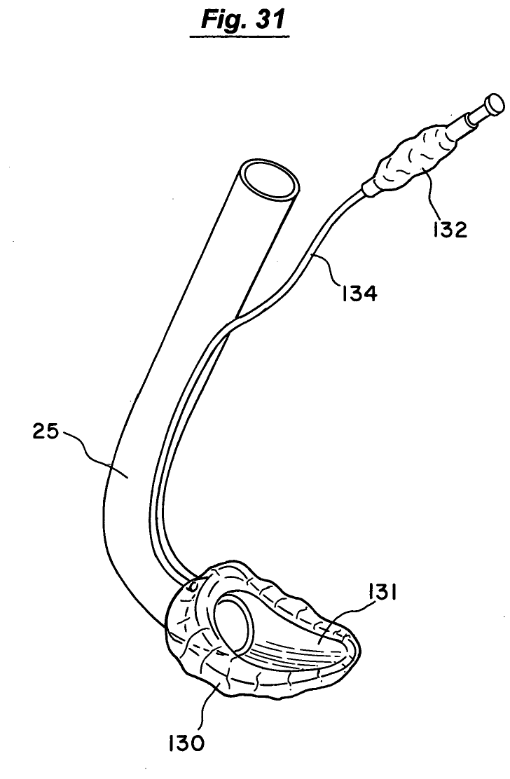

FIG. 31 is a front perspective view of yet another embodiment of the guide 25 without a separate ventilation port. The patient can be supplied with air/oxygen through a connector or cap placed in the proximal opening of the guide 25 having a ventilation port (not shown).

The following is a description of a typical method of use for the present invention. The curved distal portion of the guide 25 is first inserted into the patient's mouth and laryngopharynx 15 with the laryngeal mask 130 deflated, as shown in FIG. 33. If necessary, the ventilation port 62 can be used as a hand grip during insertion of the guide 25. FIG. 32 is a corresponding top perspective view of a patient's airway, including the larynx 18, esophagus 13, and epiglottis 13. The positions of the guide 25 and laryngeal mask 130 relative to the patient's anatomy after insertion are shown in dashed lines in FIG. 32. The lower portions of the support member 131 and laryngeal mask 130 extend into the esophagus 13. The upper portions of the support member 131 and the laryngeal mask 130 surround the glottis 19.

A protrusion 133 on the anterior portion of the distal tip of the guide 25 or support member 131 is inserted to the patient's vallecula 17 (i.e., the notch between the base of the tongue 14 and the epiglottis 13. The protrusion 133 pushes on the vallecula 17, which tends to lift the epiglottis 13 from the glottis 19 and helps to ensure patency of the patient's airway.

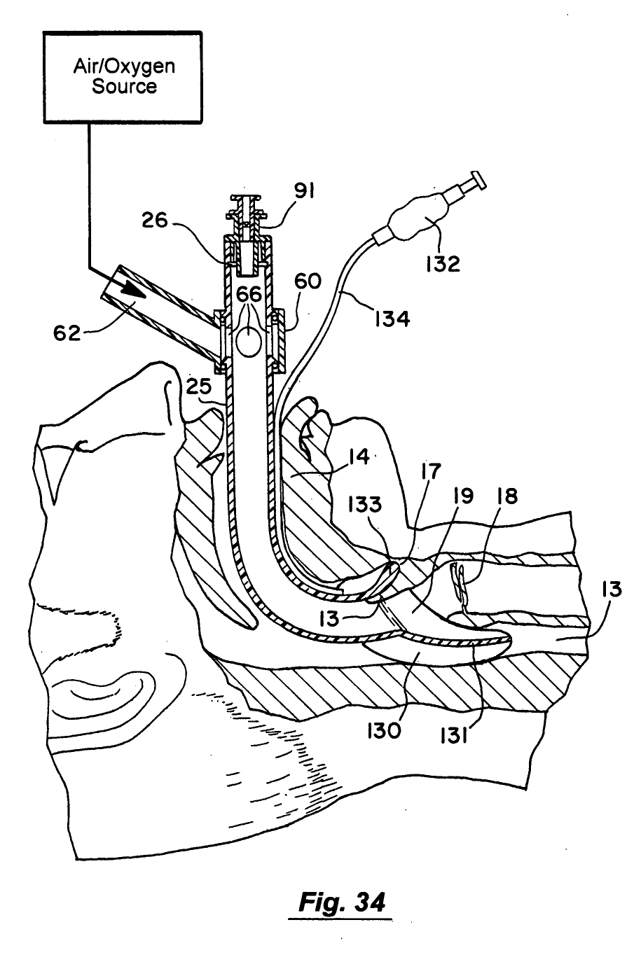

After the distal portion of the guide 25 and the laryngeal mask 130 are appropriately positioned relative to the glottis 19, the laryngeal mask 130 is inflated via the inflation tube 134 to establish a seal around the glottis 19, as depicted in FIG. 34. The lower portion 135 of the inflated laryngeal mask 130 substantially blocks the esophagus 13. The upper portion 136 of the inflated laryngeal mask 130 substantially fills the laryngopharynx 15 adjacent to the glottis 19, and thereby seals the distal opening of the guide 25 in fluid communication with the glottis. The side portions 137 and 138 (shown in FIG. 28) pinch the sides of the epiglottis 13, which also tends to lift the epiglottis 13 from the glottis 19.

If necessary, the guide cap 91 can be removed and an endoscope probe can be inserted through the proximal end of the guide 25 to enable the physician to view the insertion process and verify that the laryngeal mask 130 is correctly positioned.

Optionally, a syringe 55 containing a local anesthetic (e.g., lidocaine or xylocaine) can be connected to the luer connector on the guide cap 91 at the proximal end of the guide 25 to squirt anesthetic as the guide 25 is inserted through the patient's mouth and into the laryngopharynx 15, as shown in FIG. 35. If squirted with sufficient force, the anesthetic can be carried as far as the larynx 18 to deaden any discomfort associated with insertion of the guide 25, laryngeal mask 130, and endotracheal tube 40.

During and after insertion of the guide 25, the patient can be resuscitated by supplying air/oxygen through the ventilation port 62. For example, the flow of air can be supplied by a resuscitation bag attached to the ventilation port 62 that is manually squeezed periodically to simulate natural breathing. Alternatively, a resuscitation attachment (such as shown in FIGS. 16-18) can be removably attached to the ventilation port 62 to enable a healthcare provider to directly resuscitate the patient. As previously discussed, a series of cardiac chest compressions can also be administered to the patient's chest while artificial ventilation continues and during the intubation and resuscitation processes.

After the patient's condition has been stabilized to some degree during initial resuscitation, an endotracheal tube 40 is inserted over the distal end of an endoscope probe 30. The guide cap 91 is removed from the proximal end of the guide 25. Resuscitation, oxygenation, or artificial ventilation continue without interruption while the endoscope probe 30 and endotracheal tube 40 are advanced along the guide 25 and through the laryngeal mask 130 to a position within the trachea past the larynx 18. FIG. 36 is a cross-sectional view of the present-device during insertion of the endotracheal tube 40 and endoscope probe 30.

The seal ring 26 within the proximal end of the guide 25 has an inside diameter that is only slightly larger than the outside diameter of the endotracheal tube 40. This allows the endotracheal tube 40 to pass through the seal ring 25 and along the guide 25, but maintains a sufficiently tight fit around the endotracheal tube 40 to reduce the escape of gas through the seal. However, air/oxygen flows freely through the space between the endotracheal tube 40 and the surrounding guide 25 to maintain patient respiration.

Optionally, a removable cap 125 can be inserted into the proximal end of the endotracheal tube 40 and a stabilizer tube 120 can be attached to the endoscope probe 30, as shown in FIG. 36, to assist in advancing the endotracheal tube 40 along the guide 25. In the preferred embodiment, the stabilizer 120 is a flexible plastic tube having a C-shaped cross-section, as shown in FIG. 22, that can be readily clipped over the fiber optic probe 30 at any desired location along its length. The inside diameter of the stabilizer 120 should be selected to provide a snug, frictional fit against the exterior of the endoscope probe 30 so that the stabilizer 120 will not readily slide after it has been attached to the fiber optic probe 30. The stabilizer 120 can also be readily removed from the endoscope probe 30 by the healthcare provider for cleaning or to adjust its location on the probe 30. The stabilizer 120 should have outside dimensions sufficiently large to push the endotracheal tube 40 forward as the fiber optic probe 30 is advanced by the healthcare provider.

The proximal end of the endotracheal tube 40 can be fitted with a removable cap 125 shown in FIG. 23. This cap 125 has outside dimensions selected so that it can be inserted snugly into the proximal opening of the endotracheal tube 40 and yet is sufficiently small to pass through the guide 25, if necessary. A central passageway extends axially through the endotracheal tube cap 125 to receive the endoscope 30. The endoscope probe 30 passes freely through the cap 125. However, the cap passageway has an inside diameter smaller than the stabilizer 120, so that the stabilizer 120 will abut and push against the proximal end of the endotracheal tube 40 as the fiber optic probe 30 is advanced by the healthcare provider. This approach enables the endotracheal tube 40 and endoscope probe 30 to be advanced along the guide 25 and patient's airway as a single assembly.

The shape of the guide 25, the support member 131, and laryngeal mask 130 tend to align the distal opening of the guide 25 with the larynx 18 so that the endoscope probe 30 and endotracheal tube 40 will pass through the opening between the vocal cords. However, after emerging from the distal end of the guide 25, the direction of the distal tip of the endoscope probe 30 can be controlled by the physician. This allows the physician to carefully guide the endoscope probe 30 and endotracheal tube 40 to a position past the larynx 18 while resuscitation continues. Many conventional endoscopes include a suction channel extending the length of the fiber optic probe to its distal tip. This feature can be used to suction mucus or other secretions from the patient's airway as the endoscope/endotracheal tube assembly is inserted. Alternatively, an endoscope 30 may not be needed at all due to the anatomical alignment provided by the laryngeal mask 130, which permits “blind” intubation of the patient. In any event, the patient is being ventilated and can receive cardiac chest compressions throughout the intubation process, so the normal risks associated with intubation are not as serious if delays are encountered in completing the intubation process using the present invention.

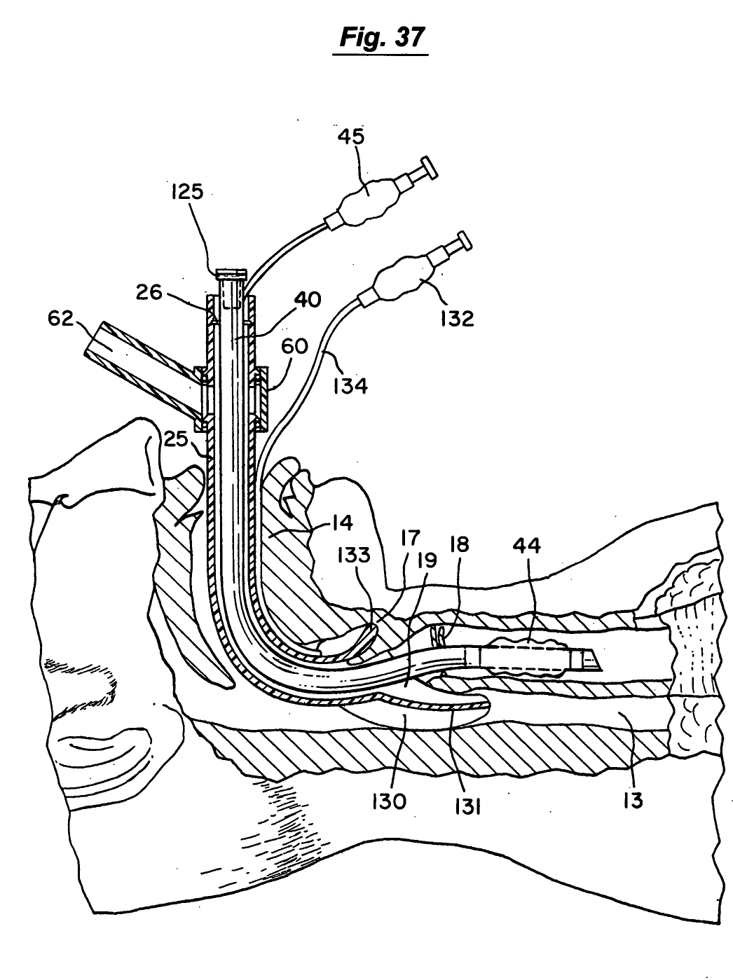

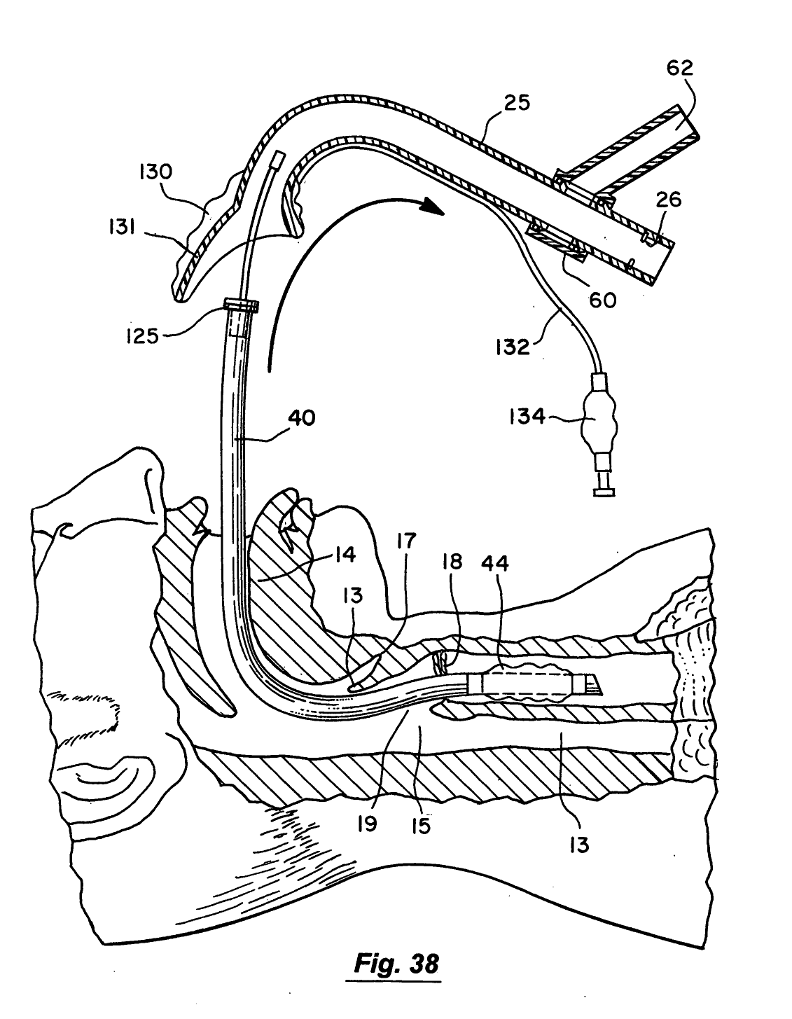

In one methodology, the endoscope probe 30 is then removed from within the endotracheal tube 40, as shown in FIG. 37. The laryngeal mask 130 is deflated and the guide 25 is removed while leaving the endotracheal tube 40 in place within the trachea, as illustrated in FIG. 38. Alternatively, the guide 25 can be left in place to serve as an oral airway and to protect the endotracheal tube 40 from being bitten by the patient's teeth. However, the laryngeal mask 130 should be deflated if the device is to be left in place in the patient's airway for an extended period time to minimize damage to the mucous lining.

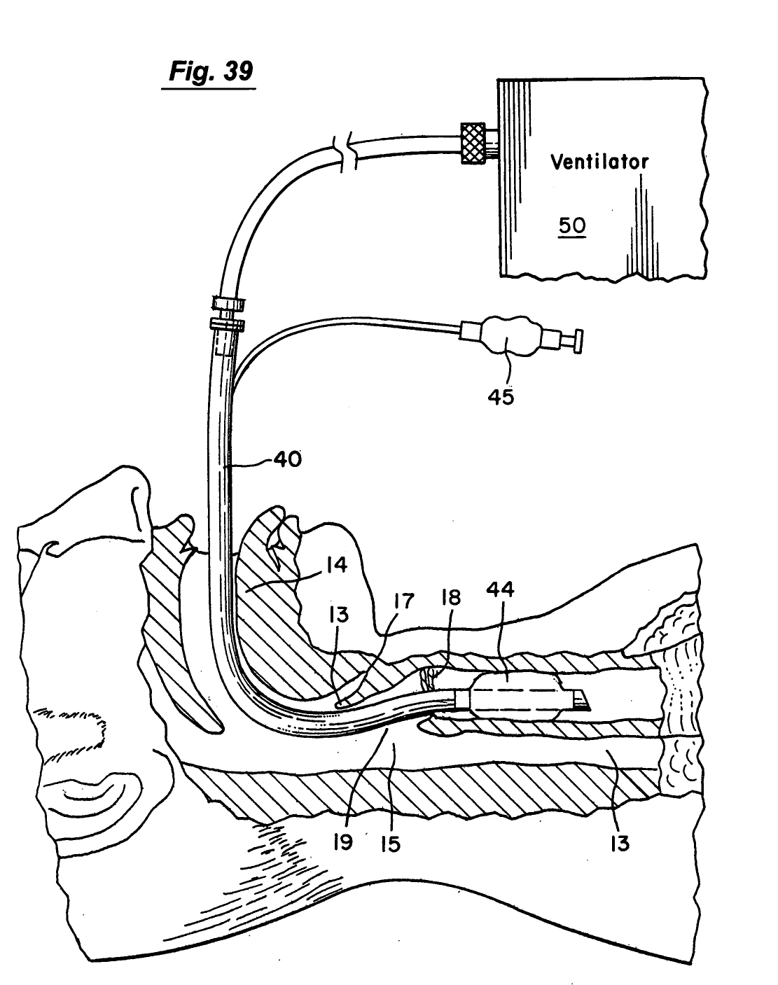

The cuff 44 on the endotracheal tube 40 is then inflated via an inflation tube and port valve 45. Finally, a ventilator 50 is connected to the proximal end of the endotracheal tube 40 to ventilate the patient, as shown in FIG. 39. Alternatively, the patient can be manually ventilated by connecting a resuscitation bag to the proximal end of the endotracheal tube 40.

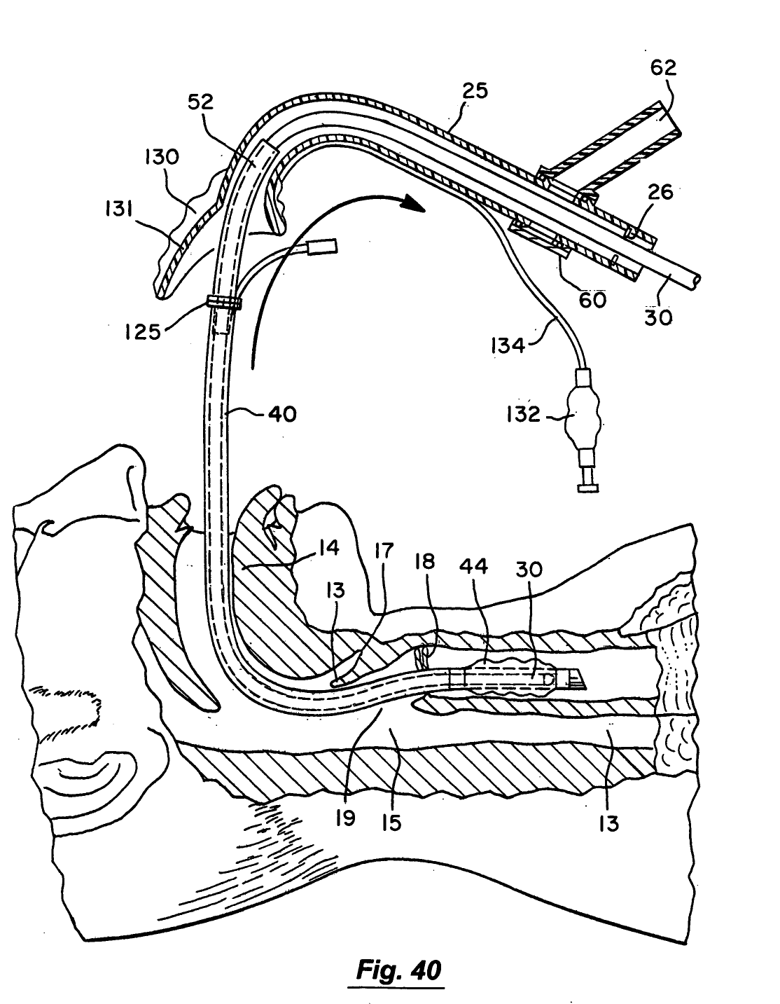

FIG. 40 depicts an alternative methodology in which the guide, 25 is withdrawn over the endoscope probe 30 while leaving the endotracheal tube 40 in place in the patient's airway. In this methodology, after the endotracheal tube 40 has been moved into position with its distal end in the trachea as illustrated in 17, the laryngeal mask 130 is deflated and the guide 25 is removed over the proximal end of the endotracheal tube 40 while leaving the endotracheal tube 40 and fiber optic probe 30 in place. Before removing the guide 25, the healthcare provider may wish to slide the stabilizer 120 a few centimeters toward the distal end of the fiber optic probe 30. This allows the endoscope 30 to be pulled back relative to the endotracheal tube 40, so that the distal tip of the endoscope 30 is located within the distal end of the endotracheal tube 40 and offers a view of both the endotracheal tube's distal tip and the patient's trachea. This enables the healthcare provider to monitor the position of the endotracheal tube 40 relative to the trachea as the guide 25 is removed, as described above.

The fiber optic probe 30 is then withdrawn from within the endotracheal tube 40 and the endotracheal tube cap 125 is removed if one is present. Finally, the patient can be ventilated via a conventional ventilator 50 connected to the endotracheal tube 40, as shown in FIG. 39.

The above disclosure sets forth a number of embodiments of the present invention. Other arrangements or embodiments, not precisely set forth, could be practiced under the teachings of the present invention and as set forth in the following claims.

1. A method for guiding insertion of an endotracheal tube into the trachea of a patient while continuing resuscitation of the patient, comprising the steps of:
inserting a curved guide through a patient's mouth and hypopharynx;
resuscitating the patient by supplying air/oxygen;
applying a series of cardiac chest compressions to the patient while resuscitation continues;
advancing the endotracheal tube along the guide into the patient's trachea while resuscitation with delivery of air/oxygen and cardiac chest compressions continue; and
ventilating the patient through the endotracheal tube.

2. The method of claim 1 wherein the guide further comprises a ventilation port for directing air/oxygen into the patient's airway.

3. The method of claim 2 further comprising the step of connecting a resuscitation attachment to the ventilation port to create a filtered flow path for a flow of air/oxygen.

4. The method of claim 1 wherein the guide further comprises a seal ring allowing passage of the endotracheal tube along the guide, but reducing the escape of air/oxygen.

5. The method of claim 1 further comprising the step of placing a face mask over the patient's mouth with the guide extending posteriorly from the face mask to allow insertion of an endotracheal tube through the face mask and along the guide.

6. The method of claim 5 wherein the face mask further comprises a ventilation port for directing air/oxygen into the patient's airway.

7. The method of claim 5 further comprising the step of removing the face mask while leaving the endotracheal tube in place within the patient's trachea.

8. The method of claim 1 further comprising the step of removing the guide while leaving the endotracheal tube in place within the patient's trachea.

9. The method of claim 1 wherein the guide further comprises a laryngeal mask extending about the distal opening of the guide,.

10. The method of claim 1 further comprising the steps of:
inserting a fiber optic probe through the endotracheal tube;
advancing the fiber optic probe and endotracheal tube along the guide into the patient's trachea while resuscitation continues; and
removing the fiber optic probe from the endotracheal tube while leaving the endotracheal tube in place within the patient's trachea.

11. The method of claim 10 wherein the fiber optic probe is advanced to a position beyond the patient's larynx.

12. The method of claim 10 further comprising the steps of:
attaching a stabilizer at a desired position on the fiber optic probe; and
inserting the fiber optic probe into the endotracheal tube until the stabilizer abuts the proximal end of the endotracheal tube.

13. The method of claim 12 wherein the stabilizer is attached to the fiber optic probe at a location so that the distal tip of the fiber optic probe extends beyond the distal tip of the endotracheal tube.

14. The method of claim 10 further comprising the steps of:
attaching a removable cap to the proximal end of the endotracheal tube prior to insertion of the fiber optic probe, said cap having a passageway to receive the fiber optic probe but reducing the escape of air/oxygen, with said passageway having an inside diameter smaller than the stabilizer; and
removing the cap from the endotracheal after the fiber optic probe is removed from the endotracheal tube and prior to ventilating the patient through the endotracheal tube.

15. A method for resuscitating a patient and guiding insertion of an endotracheal tube into the patient's trachea comprising:
placing a face mask over a patient's mouth with a removable guide extending posteriorly from the face mask allowing insertion of an endotracheal tube through the face mask and along the guide into the patient's mouth and hypopharynx;
resuscitating the patient by supplying air/oxygen;
applying a series of cardiac chest compressions to the patient while resuscitation continues;
advancing the endotracheal tube along the guide into the patient's trachea while resuscitation with delivery of air/oxygen and cardiac chest compressions continue; and
ventilating the patient through the endotracheal tube.

16. The method of claim 15 wherein the face mask further comprises a ventilation port for directing air/oxygen into the patient's airway.

17. The method of claim 15 further comprising the step of removing the face mask while leaving the endotracheal tube in place within the patient's trachea.

18. The method of claim 15 wherein the guide further comprises a seal ring allowing passage of the endotracheal tube along the guide, but reducing the escape of air/oxygen.

19. The method of claim 15 further comprising the step of removing the guide while leaving the endotracheal tube in place within the patient's trachea.

20. The method of claim 15 further comprising the steps of:
inserting a fiber optic probe through the endotracheal tube;
advancing the fiber optic probe and endotracheal tube along the guide into the patient's trachea while resuscitation continues; and
removing the fiber optic probe from the endotracheal tube while leaving the endotracheal tube in place within the patient's trachea.

21. The method of claim 20 wherein the fiber optic probe is advanced to a position beyond the patient's larynx.

22. The method of claim 20 further comprising the steps of:
attaching a stabilizer at a desired position on the fiber optic probe; and
inserting the fiber optic probe into the endotracheal tube until the stabilizer abuts the proximal end of the endotracheal tube.

23. The method of claim 22 wherein the stabilizer is attached to the fiber optic probe at a location so that the distal tip of the fiber optic probe extends beyond the distal tip of the endotracheal tube.

24. The method of claim 22 further comprising the steps of:
attaching a removable cap to the proximal end of the endotracheal tube prior to insertion of the fiber optic probe, said cap having a passageway to receive the fiber optic probe but reducing the escape of air/oxygen, with said passageway having an inside diameter smaller than the stabilizer; and
removing the cap from the endotracheal after the fiber optic probe is removed from the endotracheal tube and prior to ventilating the patient through the endotracheal tube.

25. A method for resuscitating a patient and guiding insertion of an endotracheal tube into the patient's trachea comprising:
inserting a tubular guide into a patient's mouth and hypopharynx, said guide having a curved distal portion shaped to allow insertion of an endotracheal tube through the guide into a patient's trachea, said guide further having a laryngeal mask surrounding the distal opening of the guide to substantially seal the patient's glottis about the distal opening of the guide;
supplying air/oxygen into the patient's lungs while advancing the endotracheal tube;
applying a series of cardiac chest compressions to the patient while continuing to supply air/oxygen;
advancing the endotracheal tube so that the endotracheal tube advances along the guide and into the patient's trachea while continuing to supply air/oxygen and cardiac chest compressions; and
ventilating the patient through the endotracheal tube.

26. The method of claim 25 further comprising the step of removing the guide from the endotracheal tube while leaving the endotracheal tube in place within the patient's trachea.

27. The method of claim 25 wherein the guide further comprises a seal ring allowing passage of the endotracheal tube along the guide, but reducing the escape of air/oxygen.

28. The method of claim 25 further comprising the step of inserting a fiber optic probe into the endotracheal tube, and then advancing the endotracheal tube and fiber optic probe into the patient's trachea.

29. The method of claim 28 further comprising the steps of:
attaching a stabilizer at a desired position on the fiber optic probe; and
inserting the fiber optic probe into the endotracheal tube until the stabilizer abuts the proximal end of the endotracheal tube.

30. The method of claim 29 wherein the stabilizer is attached to the fiber optic probe at a location so that the distal tip of the fiber optic probe extends beyond the distal tip of the endotracheal tube.

31. The method of claim 29 further comprising the steps of:
attaching a removable cap to the proximal end of the endotracheal tube prior to insertion of the fiber optic probe, said cap having a passageway to receive the fiber optic probe but reducing the escape of air/oxygen, with said passageway having an inside diameter smaller than the stabilizer; and
removing the cap from the endotracheal after the fiber optic probe is removed from the endotracheal tube and prior to ventilating the patient through the endotracheal tube.
